(19) United States
(12) Patent Application Publication
(10) Pub. No.: US 2012/0282643 A1
(43) Pub. Date: Nov. 8, 2012

(54) CYAN AND YELLOW FLUORESCENT COLOR VARIANTS OF SPLIT GFP

(75) Inventors: MEGHAN AILEEN LOCKARD, NEW YORK, NY (US); GEOFFREY S. WALDO, SANTA FE, NM (US)

(73) Assignee: LOS ALAMOS NATIONAL SECURITY, LLC

(21) Appl. No.: 13/101,917

(22) Filed: May 5, 2011

(51) Int. Cl. C12Q 1/02; C12N 1/00; C07K 14/435; C12N 15/12

(52) U.S. Cl. 435/29; 536/23.5; 435/243; 530/350; 530/402

(57) ABSTRACT

Disclosed herein are Split-Fluorescent proteins (SFPs) including Split-Yellow Fluorescent Proteins and Split-Cyan Fluorescent proteins. Further disclosed are methods of using SFPs. For example, methods of identifying the subcellular localization of a protein and methods of identifying the membrane topology of a membrane protein are disclosed herein.

### STATEMENT OF GOVERNMENT SUPPORT

This invention was made with government support under Contract No. DE-AC52-06NA25396 awarded by the U.S. Department of Energy, and under the National Institutes of Health's Protein Structure Initiative, grant number 5U54GM074946-4. The government has certain rights in the invention

### FIELD

Split Fluorescent Proteins (SFPs) and methods of use thereof are disclosed herein. Split-Yellow Fluorescent Proteins and Split-Cyan Fluorescent proteins are disclosed herein. Further disclosed are methods of using SFPs. For example, methods of identifying the subcellular localization of a protein and methods of identifying the membrane topology of a membrane protein involving SFPs are disclosed.

### BACKGROUND

Green Fluorescent Protein (GFP) is a fluorescent protein from the Pacific Northwest jellyfish, Aequorea victoria. Several natural and engineered GFP variants are known, including variants that exhibit altered fluorescent properties. For example, substitution of a tyrosine residue for the threonine residue at position 203 of GFP results in a fluorescent molecule with red-shifted emission characteristics, termed Yellow Fluorescent Protein (YFP). Substitution of a tryptophan residue for the tyrosine residue at position 66 of GFP results in a fluorescent molecule with blue-shifted emission characterizes, termed Cyan Fluorescent Protein (CFP). (See, for instance, U.S. Pat. Nos. 5,804,387; 6,090,919; 6,096,865; 6,054,321; 5,625,048; 5,874,304; 5,777,079; 5,968,750; 6,020,192 and 6,146,826; and published international patent application WO 99/64592).

SFPs are composed of multiple peptide fragments that individually are not fluorescent, but, when complemented, form a functional fluorescent molecule. For example, Split-Green Fluorescent Protein (Split-GFP) is a SFP. Some engineered Split-GFP molecules are self-assembling. (See, e.g., U.S. Pat. App. Pub. No. 2005/0221343 and PCT Pub. No. WO/2005/074436; Cabantous et al., Nat. Biotechnol., 23:102-107, 2005; Cabantous and Waldo, Nat. Methods, 3:845-854, 2006.)

### SUMMARY

The polypeptides, polynucleotides, and methods described herein are based on the discovery of novel polypeptide sequences comprising Split-YFP and Split-GFP molecules. As disclosed herein, introducing the conventional YFP substitution (T203Y) into Split-GFP results in a non-functional Split-YFP molecule having fluorescent properties that are not significantly distinguishable from Split-GFP. Further disclosed herein, introducing the conventional CFP substitution (Y66W) into Split-GFP results in a non-functional Split-CFP molecule lacking fluorescent properties. However, as disclosed herein, novel combinations of amino acid substitutions within Split-GFP result in functional Split-CFP and Split-YFP molecules. Thus, novel polypeptides comprising Split-YFP and Split-CFP molecules are provided herein. Methods of using the polypeptides described herein are also disclosed. Non-limiting examples of methods of using these SFPs include methods of determining the subcellular localization of a protein and methods of determining the membrane topology of a protein.

Polypeptides comprising SFP detectors are provided. In some embodiments, the polypeptides comprise a Split Fluorescent Protein (SFP) detector comprising an amino acid sequence set forth as SEQ ID NO: 23, wherein the SFP detector complements with a SFP tag to form a functional Split-Cyan Fluorescent Protein.

In other embodiments, the polypeptides comprising a Split Fluorescent Protein (SFP) detector comprising an amino acid sequence set forth as SEQ ID NO: 31, wherein the SFP detector complements with a SFP tag to form a functional Split-Yellow Fluorescent Protein.

Nucleic acid molecules encoding the disclosed polypeptides, as well as vectors and cells that include such nucleic acid molecules, are also provided.

Kits including the disclosed nucleic acid molecules, polypeptides, vectors, and/or cells are also provided.

Methods of determining the subcellular localization of a protein are provided. In some embodiments, the methods include providing within at least one host cell a first polypeptide comprising a first subcellular localization element and a first Split Fluorescent Protein (SFP) detector comprising a polypeptide comprising an amino acid sequence set forth as SEQ ID NO: 23 or SEQ ID NO: 31, wherein the first subcellular localization element localizes the first polypeptide to a first subcellular compartment; providing within the host cell a second polypeptide comprising a test protein fused to a SFP tag; and detecting fluorescence of the first SFP detector complemented with the SFP tag in the host cell, wherein the presence of fluorescence of the first SFP detector complemented with the SFP tag identifies the test protein as localized to the first subcellular compartment, thereby determining a subcellular localization of a protein.

In some embodiments, a method for detecting the localization of a test protein to one or more of a plurality of subcellular components in a cell is provided. For example, such methods include providing within the cell a polypeptide comprising the test protein and a SFP tag; providing within the cell a plurality of SFP detectors complementary to the SFP tag at least one of which is a polypeptide comprising an amino acid sequence set forth as SEQ ID NO: 23 or SEQ ID NO: 31, wherein each of the SFP detectors is capable of producing different color fluorescence upon complementation with the SFP tag and each of the SFP detectors is fused to a subcellular localization element that localizes the SFP detector to a different subcellular compartment; and detecting the various color fluorescence signals in cell, thereby detecting the localization of the test protein to one or more of the subcellular compartments.

Methods of determining the membrane topology of a membrane protein are provided. In some embodiments, such methods include providing within at least one host cell a first polypeptide comprising a first subcellular localization element and a first Split Fluorescent Protein (SFP) detector comprising a polypeptide comprising an amino acid sequence set forth as SEQ ID NO: 23 or SEQ ID NO: 31, wherein the first subcellular localization element localizes the first polypeptide to one side of a membrane of the host cell; providing within the host cell a second polypeptide comprising a test membrane protein, the N- or C-terminus of which is fused to a SFP tag; and detecting fluorescence of the first SFP detector complemented with the SFP tag in the host cell, wherein the presence of fluorescence of the first SFP detector complemented with the SFP tag in the host cell identifies the membrane orientation of the terminus of test protein fused to the SFP tag as on the same side of the membrane as the first SFP detector, thereby determining the topology of a membrane protein.

It will be further understood that the disclosed SFP variants and methods of use thereof, as well as the kits and systems disclosed herein are useful beyond the specific circumstances that are described in detail herein. The foregoing and other objects and features of the disclosure will become more apparent from the following detailed description, which proceeds with reference to the accompanying figures.

### BRIEF DESCRIPTION OF THE FIGURES

FIG. 1 shows an image of the fluorescence emitted from E. coli containing individual members of the set of Split-CFP mutants developed using the directed evolution screen described in Example 2. The identifier for individual mutants (A1-H12) is shown. Expression of the Split-CFP S1-10 fragment and the complementing S11 fragment was sequentially induced and any resulting fluorescence detected. The sequential expression protocol prevents false-positive solubility results. The excitation/emission wavelengths were 430 and 488 nm, respectively. Image capture time was four seconds.

FIG. 2 shows an image of the fluorescence emitted from E. coli containing individual members of the set of Split-YFP mutants developed using the degenerate library screen described in Example 4. The identifier for individual mutants (A1-H12) is shown (column 6 is omitted from this image). Expression of the Split-YFP S1-10 fragment and the complementing S11 fragment was sequentially induced and any resulting fluorescence detected. The sequential expression protocol prevents false-positive solubility results. The excitation/emission wavelengths were 510 and 532 nm respectively. Image capture time was 0.25 seconds.

FIG. 3 shows an image of the fluorescence emitted from multiple E. coli bacteria blobs containing individual members of the set of Split-YFP mutants developed using the degenerate library screen described in Example 4. The identifier for individual mutants (A1-H12) is shown. Expression of the Split-YFP S1-10 fragment and the complementing S11 fragment was sequentially induced and any resulting fluorescence detected. The sequential expression protocol prevents false-positive solubility results. The excitation/emission wavelengths were 488 and 510 nm respectively. Image capture time was 0.25 seconds.

FIG. 4 shows an image of the fluorescence emitted from E. coli bacteria blobs containing optima from the set of Split-CFP mutants developed using the directed evolution screen described in Example 2. Expression and detection were performed as above. Specific substitutions in relation to GFP S-1-10 (SEQ ID NO: 4) are shown. The individual mutants shown are indicated in the figure. The excitation/emission wavelengths were 430 and 488 nm, respectively. Image capture time was four seconds.

FIG. 5 shows an image of the yellow and green fluorescence emitted from multiple E. coli bacteria blobs containing individual members of the set of Split-YFP mutants developed using the directed evolution screen described in Example 4. Expression and detection were performed as above. Specific substitutions in relation to GFP S-1-10 (SEQ ID NO: 4) are shown. The excitation/emission wavelengths were 510 and 532 nm for the yellow channel, respectively, and 488 and 510 for the green channel, respectively. Image capture time was 0.25 seconds.

FIG. 6 shows a graph of a XY plot of the normalized initial rate and final fluorescence measurements for the Split-CFP S-10 kinetic experiments for each of the Split-CFP S-10 substitutions described in Example 2. The two points labeled “A1” and “C1” correspond to the measurements of the Split-CFP optima described in Example 2.

### SEQUENCES

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing is submitted as an ASCII text file, created on Apr. 12, 2011, 45 KB, which is incorporated by reference herein.

SEQ ID NO: 1 is an exemplary cDNA sequence encoding the polypeptide of SEQ ID NO: 2.

SEQ ID NO: 2 is the amino acid sequence of GFP superfolder 1-10.

SEQ ID NO: 3 is an exemplary cDNA sequence encoding the polypeptide of SEQ ID NO: 4.

SEQ ID NO: 4 is the amino acid sequence of GFP 1-10 OPT (additional mutations vs. superfolder: N39I, T105K, E111V, I128T, K166T, 1167V, S205T).

SEQ ID NO: 5 is an exemplary cDNA sequence encoding the polypeptide of SEQ ID NO: 6.

SEQ ID NO: 6 is the amino acid sequence of GFP 1-10 A4 (additional mutations versus Superfolder GFP: R80Q, S99Y, T105N, E111V, I128T, K166T, E172V, S205T).

SEQ ID NO: 7 is an exemplary cDNA sequence encoding the polypeptide of SEQ ID NO: 8.

SEQ ID NO: 8 is the amino acid sequence of GFP S11 214-238.

SEQ ID NO: 9 is an exemplary cDNA sequence encoding the polypeptide of SEQ ID NO: 10.

SEQ ID NO: 10 is the amino acid sequence of GFP S11 214-230. SEQ ID NO: 11 is an exemplary cDNA sequence encoding the polypeptide of SEQ ID NO: 12.

SEQ ID NO: 12 is the amino acid sequence of GFP S11 M1 amino acid sequence (additional mutation versus wt: L221H).

SEQ ID NO: 13 is an exemplary cDNA sequence encoding the polypeptide of SEQ ID NO: 14.

SEQ ID NO: 14 is the amino acid sequence of GFP S11 M2 (additional mutations versus GFP S11 wt: L221H, F2235, T225N).

SEQ ID NO: 15 is an exemplary cDNA sequence encoding the polypeptide of SEQ ID NO: 16.

SEQ ID NO: 16 is the amino acid sequence of GFP S11 M3 (additional mutations versus GFP S11 wt: L221H, F223Y, T225N).

SEQ ID NO: 17 is the amino acid sequence of Split-CFP S1-10 Y66W.

SEQ ID NO: 18 is the amino acid sequence of Split-CFP S1-10 Y66W, H148D, T205S.

SEQ ID NO: 19 is the amino acid sequence of Split-CFP S1-10 D19E, D21E, Y66W, H148D, T2055.

SEQ ID NO: 20 is the amino acid sequence of Split-CFP S1-10 OPT1 (D19E, D21E, Y66W, E124V, H148D, T2055).

SEQ ID NO: 21 is the amino acid sequence of Split-CFP S1-10 OPT2 (D19E, D21E, Y66W, H148D, V1671, T2055).

SEQ ID NO: 22 is the amino acid sequence of Split-CFP S1-10 consensus sequence 1.

SEQ ID NO: 23 is the amino acid sequence of Split-CFP S1-10 consensus sequence 2.

SEQ ID NO: 24 is the amino acid sequence of Split-YFP S1-10 T203Y.

SEQ ID NO: 25 is the amino acid sequence of Split-YFP S1-10 OPT1 (T65L, T203Y, T2055).

SEQ ID NO: 26 is the amino acid sequence of Split-YFP S1-10 OPT2 (T65G, T203Y, T2055).

SEQ ID NO: 27 is the amino acid sequence of Split-YFP S1-10 OPT3 (T203Y, T2055).

SEQ ID NO: 28 is the amino acid sequence of Split-YFP S1-10 (T65A, T203Y, T2055).

SEQ ID NO: 29 is the amino acid sequence of Split-YFP S1-10 (T203Y, T205A).

SEQ ID NO: 30 is the amino acid sequence of Split-YFP S1-10 consensus sequence 1.

SEQ ID NO: 31 is the amino acid sequence of Split YFP S1-10 consensus 2.

SEQ ID NO: 32 is the amino acid sequence of Nuclear localization signal (NLS) of the simian virus 40 large T-antigen.

SEQ ID NO: 33 is an exemplary cDNA sequence the polypeptide of SEQ ID NO: 32.

SEQ ID NO: 34 is the amino acid sequence of the N-terminal 81 amino acids of human beta 1,4-galactosyltransferase (GT).

SEQ ID NO: 35 is an exemplary cDNA sequence encoding the polypeptide of SEQ ID NO: 34.

SEQ ID NO: 36 is the amino acid sequence of the mitochondria targeting sequence derived from the precursor of subunit VIII of human cytochrome C oxidase.

SEQ ID NO: 37 is an exemplary cDNA sequence encoding the polypeptide of SEQ ID NO: 36.

SEQ ID NO: 38 is the amino acid sequence of the ER targeting sequence of calreticulin.

SEQ ID NO: 39 is an exemplary cDNA sequence encoding the polypeptide of SEQ ID NO: 38.

TABLE OF SEQUENCES
SEQ ID NO: 1
GFP superfolder 1-10 nucleotide sequence:
ATGAGCAAAGGAGAAGAACTTTTCACTGGAGTTGTCCCAATTCTTGTTGA
ATTAGATGGTGATGTTAATGGGCACAAATTTTCTGTCAGAGGAGAGGGTG
AAGGTGATGCTACAAACGGAAAACTCACCCTTAAATTTATTTGCACTACT
GGAAAACTACCTGTTCCATGGCCAACACTTGTCACTACTCTGACCTATGG
TGTTCAATGCTTTTCCCGTTATCCGGATCACATGAAACGGCATGACTTTT
TCAAGAGTGCCATGCCCGAAGGTTATGTACAGGAACGCACTATATCTTTC
AAAGATGACGGGACCTACAAGACGCGTGCTGAAGTCAAGTTTGAAGGTGA
TACCCTTGTTAATCGTATCGAGTTAAAAGGTATTGATTTTAAAGAAGATG
GAAACATTCTCGGACACAAACTCGAGTACAACTTTAACTCACACAATGTA
TACATCACGGCAGACAAACAAAAGAATGGAATCAAAGCTAACTTCAAAAT
TCGCCACAACGTTGAAGATGGTTCCGTTCAACTAGCAGACCATTATCAAC
AAAATACTCCAATTGGCGATGGCCCTGTCCTTTTACCAGACAACCATTAC
CTGTCGACACAATCTGTCCTTTCGAAAGATCCCAACGAAAAGCTAA
SEQ ID NO: 2
GFP superfolder 1-10 amino acid sequence:
MSKGEELFTGVVPILVELDGDVNGHKFSVRGEGEGDATNGKLTLKFICTT
GKLPVPWPTLVTTLTYGVQCFSRYPDHMKRHDFFKSAMPEGYVQERTISF
KDDGTYKTRAEVKFEGDTLVNRIELKGIDFKEDGNILGHKLEYNFNSHNV
YITADKQKNGIKANFKIRHNVEDGSVQLADHYQQNTPIGDGPVLLPDNHY
LSTQSVLSKDPNEK
SEQ ID NO: 3
GFP 1-10 OPT nucleotide sequence:
ATGAGCAAAGGAGAAGAACTTTTCACTGGAGTTGTCCCAATTCTTGTTGA
ATTAGATGGTGATGTTAATGGGCACAAATTTTCTGTCAGAGGAGAGGGTG
AAGGTGATGCTACAATCGGAAAACTCACCCTTAAATTTATTTGCACTACT
GGAAAACTACCTGTTCCATGGCCAACACTTGTCACTACTCTGACCTATGG
TGTTCAATGCTTTTCCCGTTATCCGGATCACATGAAAAGGCATGACTTTT
TCAAGAGTGCCATGCCCGAAGGTTATGTACAGGAACGCACTATATCTTTC
AAAGATGACGGGAAATACAAGACGCGTGCTGTAGTCAAGTTTGAAGGTGA
TACCCTTGTTAATCGTATCGAGTTAAAGGGTACTGATTTTAAAGAAGATG
GAAACATTCTCGGACACAAACTCGAGTACAACTTTAACTCACACAATGTA
TACATCACGGCAGACAAACAAAAGAATGGAATCAAAGCTAACTTCACAGT
TCGCCACAACGTTGAAGATGGTTCCGTTCAACTAGCAGACCATTATCAAC
AAAATACTCCAATTGGCGATGGCCCTGTCCTTTTACCAGACAACCATTAC
CTGTCGACACAAACTGTCCTTTCGAAAGATCCCAACGAAAAGGGTACCTA
A
SEQ ID NO: 4
GFP 1-10 OPT amino acid sequence (additional
mutations vs. superfolder: N39I, T105K, E111V,
I128T, K166T, I167V, 5205T):
MSKGEELFTGVVPILVELDGDVNGHKFSVRGEGEGDATIGKLTLKFICTT
GKLPVPWPTLVTTLTYGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSTQTVLSKDPNEKGT
SEQ ID NO: 5
GFP 1-10 A4 nucleotide sequence:
ATGAGCAAAGGAGAAGAACTTTTCACTGGAGTTGTCCCAATTCTTGTTGA
ATTAGATGGAGATGTTAATGGGCACAAATTTTCTGTCAGAGGAGAGGGTG
AAGGTGATGCTACAAACGGAAAACTCACCCTTAAATTCATTTGCACTACT
GGAAAACTACCTGTTCCATGGCCAACGCTTGTCACTACTCTGACCTATGG
TGTTCAATGCTTTTCCCGTTATCCGGATCACATGAAACAGCATGACTTTT
TCAAGAGTGCCATGCCCGAAGGTTATGTACAGGAACGCACTATATATTTC
AAAGATGACGGGAACTACAAGACGCGTGCTGTAGTCAAGTTTGAAGGTGA
TACCCTTGTTAATCGTATCGAGTTAAAGGGTACTGATTTTAAAGAAGATG
GAAACATTCTCGGACACAAACTCGAGTACAACTTTAACTCACACAATGTA
TATATCACGGCAGACAAACAAAAGAATGGAATCAAAGCTAACTTCACAAT
TCGCCACAACGTTGTAGATGGTTCCGTTCAACTAGCAGACCATTATCAAC
AAAATACTCCAATTGGCGATGGCCCTGTCCTTTTACCAGACAACCATTAC
TTGTCGACACAAACTGTCCTTTCGAAAGATCCCAACGAAAAGGGTACCTA
A
SEQ ID NO: 6
GFP 1-10 A4 amino acid sequence (additional
mutations versus Superfolder GFP: R80Q,
S99Y, T105N, E111V, I128T, K166T, E172V,
D205T):
MSKGEELFTGVVPILVELDGDVNGHKFSVRGEGEGDATNGKLTLKFICTT
GKLPVPWPTLVTTLTYGVQCFSRYPDHMKQHDFFKSAMPEGYVQERTIYF
KDDGNYKTRAVVKFEGDTLVNRIELKGTDFKEDGNILGHKLEYNFNSHNV
YITADKQKNGIKANFTIRHNVVDGSVQLADHYQQNTPIGDGPVLLPDNHY
LSTQTVLSKDPNEKGT
SEQ ID NO: 7
GFP S11 214-238 nucleotide sequence:
AAGCGTGACCACATGGTCCTTCTTGAGTTTGTAACTGCTGCTGGGATTAC
ACATGGCATGGATGAGCTCTACAAAGGTACCTAA
SEQ ID NO: 8
GFP S11 214-238 amino acid sequence:
KRDHMVLLEFVTAAGITHGMDELYKGT
SEQ ID NO: 9
GFP S11 214-230 nucleotide sequence:
AAGCGTGACCACATGGTCCTTCTTGAGTTTGTAACTGCTGCTGGGATTAC
AGGTACCTAA
SEQ ID NO: 10
GFP S11 214-230 amino acid sequence:
KRDHMVLLEFVTAAGITGT
SEQ ID NO: 11
GFP S11 M1 nucleotide sequence:
AAGCGTGACCACATGGTCCTTCATGAGTTTGTAACTGCTGCTGGGATTAC
AGGTACCTAA
SEQ ID NO: 12
GFP S11 M1 amino acid sequence (additional
mutation versus wt: L221H):
KRDHMVLHEFVTAAGITGT
SEQ ID NO: 13
GFP S11 M2 nucleotide sequence:
AAGCGTGACCACATGGTCCTTCATGAGTCTGTAAATGCTGCTGGGGGTAC
CTAA
SEQ ID NO: 14
GFP S11 M2 amino acid sequence: (additional
mutations versus GFP S11 wt: L221H, F2235,
T225N):
KRDHMVLHESVNAAGGT
SEQ ID NO: 15
GFP S11 M3 nucleotide sequence:
CGTGACCACATGGTCCTTCATGAGTCTGTAAATGCTGCTGGGATTACATA
A
SEQ ID NO: 16
GFP S11 M3 amino acid sequence (additional
mutations versus GFP S11 wt: L221H, F223Y,
T225N):
RDHMVLHEYVNAAGIT
SEQ ID NO: 17
Split-CFP S1-10 Y66W (nonfunctional):
MSKGEELFTGVVPILVELDGDVNGHKFSVRGEGEGDATIGKLTLKFICTT
GKLPVPWPTLVTTLTWGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSTQTSVLSKDPNEKGS
SEQ ID NO: 18
Split-CFP S1-10 (Y66W, H148D, T205S):
MSKGEELFTGVVPILVELDGDVNGHKFSVRGEGEGDATIGKLTLKFICTT
GKLPVPWPTLVTTLTWGVQCFSRYPDHMKRHDFFKSAMPEGYVQERTISF
KDDGKYKTRAVVKFEGDTLVNRIELKGTDFKEDGNILGHKLEYNFNSDNV
YITADKQKNGIKANFTVRHNVEDGSVQLADHYQQNTPIGDGPVLLPDNHY
LSTQSVLSKDPNEKGS
SEQ ID NO: 19
Split-CFP S1-10 (D19E, D21E, Y66W, H148D,
T205S):
MSKGEELFTGVVPILVELEGEVNGHKFSVRGEGEGDATIGKLTLKFICTT
GKLPVPWPTLVTTLTWGVQCFSRYPDHMKRHDFFKSAMPEGYVQERTISF
KDDGKYKTRAVVKFEGDTLVNRIELKGTDFKEDGNILGHKLEYNFNSDNV
YITADKQKNGIKANFTVRHNVEDGSVQLADHYQQNTPIGDGPVLLPDNHY
LSTQSVLSKDPNEKGS
SEQ ID NO: 20
Split-CFP S1-10 OPT1 (D19E, D21E, Y66W,
E124V, H148D, T205S):
MSKGEELFTGVVPILVELEGEVNGHKFSVRGEGEGDATIGKLTLKFICTT
GKLPVPWPTLVTTLTWGVQCFSRYPDHMKRHDFFKSAMPEGYVQERTISF
KDDGKYKTRAVVKFEGDTLVNRIVLKGTDFKEDGNILGHKLEYNFNSDNV
YITADKQKNGIKANFTVRHNVEDGSVQLADHYQQNTPIGDGPVLLPDNHY
LSTQSVLSKDPNEKGS
SEQ ID NO: 21
Split-CFP S1-10 OPT 2 (D19E, D21E, Y66W,
H148D, V167I, T205S):
MSKGEELFTGVVPILVELEGEVNGHKFSVRGEGEGDATIGKLTLKFICTT
GKLPVPWPTLVTTLTWGVQCFSRYPDHMKRHDFFKSAMPEGYVQERTISF
KDDGKYKTRAVVKFEGDTLVNRIELKGTDFKEDGNILGHKLEYNFNSDNV
YITADKQKNGIKANFTIRHNVEDGSVQLADHYQQNTPIGDGPVLLPDNHY
LSTQSVLSKDPNEKGS
SEQ ID NO: 22
Split-CFP S1-10 consensus sequence 1:
MSKGEELFTGVVPILX₁[16]ELX₂[19]GX₃[21]VNGHKFSVRGEGEG
DATIGKLTLKFICTTGKLPVPWPTLVTTLTWGVQCFSRYPDHMKRHDFFK
SAMPEGYVQERTIX₄[99]FKDDGKYKTRAVVKFEGDTLVNRIX₅[124]
LKGTDFKEDGNILGHKLEYNFNSDNVYITADKQKNGIKANFTX₆[167]R
HNVEDGSVQLADHYQQNTPIGDGPVLLPDNHYLSTQSVLSKDPNEKGS
wherein X₁ is V or I, X₂ is D or E,
X₃ is D, E or N, X₄ is S or T, X₅ is
E or V, and X₆ is V or I.
SEQ ID NO: 23
Split-CFP S1-10 consensus sequence 2:
MSKGEELFTGVVPILVELEGEVNGHKFSVRGEGEGDATIGKLTLKFICTT
GKLPVPWPTLVTTLTWGVQCFSRYPDHMKRHDFFKSAMPEGYVQERTISF
KDDGKYKTRAVVKFEGDTLVNRIX₁[124]LKGTDFKEDGNILGHKLEYN
FNSDNVYITADKQKNGIKANFTX₂[167]RHNVEDGSVQLADHYQQNTPI
GDGPVLLPDNHYLSTQSVLSKDPNEKGS
wherein X₁ is E or V, and X₂ is V or I.
SEQ ID NO: 24
Split-YFP S1-10 (T203Y):
MSKGEELFTGVVPILVELDGDVNGHKFSVRGEGEGDATIGKLTLKFICTT
GKLPVPWPTLVTTLTYGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSYQTVLSKDPNEKGS
SEQ ID NO: 25
Split-YFP S1-10 OPT1 (T65L, T203Y, T205S):
MSKGEELFTGVVPILVELDGDVNGHKFSVRGEGEGDATIGKLTLKFICTT
GKLPVPWPTLVTTLLYGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSYQSVLSKDPNEKGS
SEQ ID NO: 26
Split-YFP S1-10 OPT2 (T65G, T203Y, T205S):
MSKGEELFTGVVPILVELDGDVNGHKFSVRGEGEGDATIGKLTLKFICTT
GKLPVPWPTLVTTLGYGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSYQSVLSKDPNEKGS
SEQ ID NO: 27
Split-YFP S1-10 OPT3 (T203Y, T205S):
MSKGEELFTGVVPILVELDGDVNGHKFSVRGEGEGDATIGKLTLKFICTT
GKLPVPWPTLVTTLTYGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSYQSVLSKDPNEKGS
SEQ ID NO: 28
Split-YFP S1-10 (T65A, T203Y, T205S):
MSKGEELFTGVVPILVELDGDVNGHKFSVRGEGEGDATIGKLTLKFICTT
GKLPVPWPTLVTTLAYGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSYQSVLSKDPNEKGS
SEQ ID NO: 29
Split-YFP S1-10 (T203Y, T205A):
MSKGEELFTGVVPILVELDGDVNGHKFSVRGEGEGDATIGKLTLKFICTT
GKLPVPWPTLVTTLTYGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSYQAVLSKDPNEKGS
SEQ ID NO: 30
Split-YFP S1-10 consensus 1:
MSKGEELFX₁[9]GVVPILVELDGDVNGHKFSVRGEGEGDATIGKLTLKF
ICTTGKLPVPWPTLVTTLX₂[65]YGVQX₃[70]FSRYPDHMKX₄[80]H
DFFKSAMPEGYVQERTIX₅[99]FKDDGKYKTRAVVKFEGDTLVNRIELK
GTDFKEDGNILGHKLEYNFNSHNVYITADKQKNGIKANFTVRHNVEDGS
X₆[176]QLADHYQQNTPIGDGX₇[192]VLLPDNHX₈[200]LSY
[203]X₉[204]X₁₀[205]VLSKX₁₁[210]PNEKGS
wherein X₁ is T or N, X₂ is T, L, G or A,
X₃ is C or S, X₄ is R or K, X₅ is S or F,
and X₆ is V or I, X₇ is P or H, X₈ is Y or
F, X₉ is Q, H or E, X₁₀ is T, S or A and
X₁₁ is D or V.
SEQ ID NO: 31
Split YFP S1-10 consensus 2:
MSKGEELFTGVVPILVELDGDVNGHKFSVRGEGEGDATIGKLTLKFICTT
GKLPVPWPTLVTTLX₁[65]YGVQCFSRYPDHMKRHDFFKSAMPEGYVQE
RTISFKDDGKYKTRAVVKFEGDTLVNRIELKGTDFKEDGNILGHKLEYNF
NSHNVYITADKQKNGIKANFTVRHNVEDGSVQLADHYQQNTPIGDGPVLL
PDNHYLSY[203]QX₂[205]VLSKDPNEKGS
wherein X₁ is T, L, G or A and X2 is S,
or X₁ is T and X₂ is S or A.
SEQ ID NO: 32
Nuclear localization signal (NLS) of the
simian virus 40 large T-antigen:
SKKEEKGRSKKEEKGRSKKEEKGRIHRI
SEQ ID NO: 33
Exemplary nucleotide sequence encoding
the polypeptide of SEQ ID NO: 32:
TCCAAAAAAGAAGAGAAAGGTAGATCCAAAAAAGAAGAGAAAGGTAGATC
CAAAAAAGAAGAGAAAGGTAGGATCCACCGGATCTAG
SEQ ID NO: 34
N-terminal 81 amino acids of human
beta 1,4-galactosyltransferase (GT):
MRLREPLLSGSAAMPGASLQRACRLLVAVCALHLGVTLVYYLAGRDLSRL
PQLVGVSTPLQGGSNSAAAIGQSSGELRTGGAKDPPVAT
SEQ ID NO: 35
Exemplary nucleotide sequence encoding
the polypeptide of SEQ ID NO: 34:
ATGAGGCTTCGGGAGCCGCTCCTGAGCGGCAGCGCCGCGATGCCAGGCG
CGTCCCTACAGCGGGCCTGCCGCCTGCTCGTGGCCGTCTGCGCTCTGCA
CCTTGGCGTCACCCTCGTTTACTACCTGGCTGGCCGCGACCTGAGCCGC
CTGCCCCAACTGGTCGGAGTCTCCACACCGCTGCAGGGCGGCTCGAACA
GTGCCGCCGCCATCGGGCAGTCCTCCGGGGAGCTCCGGACCGGAGGGGC
CAAGGATCCACCGGTCGCCACC
SEQ ID NO: 36
Mitochondria targeting sequence derived
from the precursor of subunit VIII of human
cytochrome C oxidase:
MSVLTPLLLRGLTGSARRLPVPRAKIHSLGDPPVAT
SEQ ID NO: 37
Exemplary nucleotide sequence encoding
the polypeptide of SEQ ID NO: 36:
ATGTCCGTCCTGACGCCGCTGCTGCTGCGGGGCTTGACAGGCTCGGCCCG
GCGGCTCCCAGTGCCGCGCGCCAAGATCCATTCGTTGGGGGATCCACCGG
TCGCCACC
SEQ ID NO: 38
ER targeting sequence of calreticulin:
MLLSVPLLLGLLGLAVAV
SEQ ID NO: 39
Exemplary nucleotide sequence encoding
the polypeptide of SEQ ID NO: 38:
ATGCTGCTATCCGTGCCGTTGCTGCTCGGCCTCCTCGGCCTGGCCGTCGC
CGTG

### DETAILED DESCRIPTION

### I. Terms and Abbreviations

cDNA Complementary DNA

CFP Cyan fluorescent protein

dsDNA Double-stranded DNA

DNA Deoxyribonucleic acid

GFP Green Fluorescent Protein

IPTG Isopropyl β-D-1-thiogalactopyranoside

LB agar Luria-Bertani agar

MCS Multiple cloning site

NLS Nuclear Localization Sequence

ORF Open reading frame

PBS Phosphate-buffered saline

PCR Polymerase chain reaction

RMSD Root mean square deviation

GFP S1-9 Beta strands 1-9 of GFP

GFP S1-10 Beta strands 1-10 of GFP

GFP S10 Beta strand 10 of GFP

GFP S11 Beta strand 11 of GFP

SDS-PAGE Sodium dodecyl sulfate-polyacrylamide gel electrophoresis

SFP Split Fluorescent Protein

Tet Tetracycline

TNG Tris-sodium-glycerol (buffer)

WB Western blot

YFP Yellow fluorescent protein

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid” includes single or plural nucleic acids and is considered equivalent to the phrase “comprising at least one nucleic acid.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to October 2010); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999; Loudon, Organic Chemistry, Fourth Edition, New York: Oxford University Press, 2002; Smith and March, March's Advanced Organic Chemistry: Reactions, Mechanisms, and Structure, Fifth Edition, Wiley-Interscience, 2001; Chalfie and Kain (Eds), Green Fluorescent Protein: Properties, Applications and Protocols, First Edition, Wiley-Liss, 1998; or Hicks (ed.), Green Fluorescent Protein: Applications & Protocols, First Edition, Humana Press, 2001.

All publications, patent applications, patents, and other references mentioned herein are incorporated by reference in their entirety. All sequences referred to by GenBank Accession numbers herein are incorporated by reference as they appeared in the database on Mar. 24, 2011. In case of conflict, the present specification, including explanations of terms, will control. In addition, the materials, methods, and examples are illustrative only and not intended to be limiting.

Amino Acid:

Naturally occurring or synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

Binding:

A specific interaction between two molecules. For example, binding can occur between a two fragments of a split fluorescent molecule (e.g., GFP S1-10 and GFP S11), or between a receptor and a particular ligand. Binding can be specific and selective, so that one molecule is bound preferentially when compared to another molecule. In one example, specific binding is identified by a disassociation constant (K_{d}) of an agent for a particular protein or class of proteins, compared to the K_{d} for one or more other cellular proteins. In another example, specific binding of an antagonist for a receptor is identified by an inhibitory concentration (IC₅₀).

cDNA (Complementary DNA):

A piece of DNA lacking internal, non-coding segments (introns) and transcriptional regulatory sequences. cDNA may also contain untranslated regions (UTRs) that are involved in translational control in the corresponding RNA molecule. cDNA can be synthesized in the laboratory by reverse transcription from messenger RNA extracted from cells.

DNA (Deoxyribonucleic Acid):

DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached.

Unless otherwise specified, any reference to a DNA molecule is intended to include the reverse complement of that DNA molecule. Except where single-strandedness is required by context, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Expression:

The process by which the coded information of a gene is converted into an operational, non-operational, or structural part of a cell, such as the synthesis of a protein.

Flow Cytometry:

A method for detecting and counting microscopic particles (e.g., cells) by suspending them in a stream of fluid and passing them by an electronic detection apparatus. Flow cytometry methods are well known to the skilled artisan and apparatuses for performing flow cytometry are commercially available. Fluorescence-activated cell sorting is a flow cytometry method for detecting and sorting cells on the basis of immunofluorescence. See, e.g., Robinson et al. (Eds.), Current Protocols in Cytometry, Wiley-Liss Pub, 2011.

Fluorescent Protein:

A protein or protein complex that has the ability to emit light of a particular wavelength (emission wavelength) when exposed to light of another wavelength (excitation wavelength). Non-limiting examples of fluorescent proteins include the green fluorescent protein (GFP; see, for instance, GenBank Accession Number M62654) from the Pacific Northwest jellyfish, Aequorea victoria and natural and engineered variants thereof (see, for instance, U.S. Pat. Nos. 5,804,387; 6,090,919; 6,096,865; 6,054,321; 5,625,048; 5,874,304; 5,777,079; 5,968,750; 6,020,192; and 6,146,826; and published international patent application WO 99/64592). Other examples include Split-GFP, Split-YFP (described herein), Split-CFP (described herein) and Split-GFP variants, folding variants of GFP (e.g., more soluble versions, superfolder versions), spectral variants of GFP which have a different fluorescence spectrum (e.g., YFP, CFP), and GFP-like fluorescent proteins (e.g., DsRed; and DsRed variants, including DsRed1, DsRed2 (see, e.g., Matz et al., Nat. Biotechnol., 17:969-973, 1999). Fluorescent proteins with distinct excitation and emission properties are familiar to the skilled artisan; for example, functional GFPs, CFPs and YFPs comprise distinct excitation and emission properties. (see. e.g., Tsien, Annu. Rev. Biochem., 67:509-544, 1998.)

Fused:

Linkage by covalent bonding.

Host Cell or Recombinant Host Cell:

A cell that has been genetically altered, or is capable of being genetically altered by introduction of an exogenous polynucleotide, such as a recombinant plasmid or vector. Typically, a host cell is a cell in which a vector can be propagated and its DNA expressed. The cell may be prokaryotic or eukaryotic. For example, the host cell may be a bacteria cell, including an E. coli cell. “Host cell” also includes a colony of cells, for example, a colony of E. coli cells. Thus, “contacting a host cell” and “incubating a host cell” include contacting a colony of host cells or incubating a colony of host cells. The term also includes any progeny of the subject host cell. It is understood that all progeny may not be identical to the parental cell since there may be mutations that occur during replication. However, such progeny are included when the term “host cell” is used. A host cell encompasses material inside the outermost cell membrane, the outermost cell membrane itself and material fused or attached to the outermost cell membrane. In the case of a cell having a cell wall, the outermost cell membrane is the cell wall. Thus, the phase “within a host cell” includes material inside the outermost cell membrane, the outermost cell membrane itself and material fused or attached to the outermost cell membrane.

Isolated:

A biological component (such as a host cell, nucleic acid molecule or polypeptide) that has been substantially separated or purified away from other biological components in the medium, cell or organism in which the component occurs. The term isolated does not require absolute purity. Nucleic acids and proteins that have been isolated include nucleic acids and proteins purified by standard purification methods. The term also embraces nucleic acids and proteins prepared by recombinant expression in a host cell, as well as chemically synthesized nucleic acids.

Multiple Cloning Site (MCS):

A region of DNA containing a series of restriction enzyme recognition sequences. Typically, the restriction sites are only present once in the MCS. Vectors and plasmids used for cloning and expression typically contain a MCS to facilitate insertion of a heterologous nucleic acid sequence, such as the coding sequence of a gene of interest. In some embodiments, a MCS comprising at least two, at least three, at least four, at least five or at least six restriction enzyme recognition sites. The restriction sites may be immediately adjacent, they may overlap, there may be one or more nucleic acids between the sites, or any combination thereof.

Nucleic Acid Molecule:

A polymeric form of nucleotides, which may include both sense and anti-sense strands of RNA, cDNA, genomic DNA, and synthetic forms and mixed polymers thereof. A nucleotide refers to a ribonucleotide, deoxynucleotide or a modified form of either type of nucleotide. The phrase nucleic acid molecule as used herein is synonymous with nucleic acid and polynucleotide. A nucleic acid molecule is usually at least six bases in length, unless otherwise specified. The term includes single- and double-stranded forms. The term includes both linear and circular (plasmid) forms. A polynucleotide may include either or both naturally occurring and modified nucleotides linked together by naturally occurring nucleotide linkages and/or non-naturally occurring chemical bonds and/or linkers.

Nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, polypeptides), intercalators (for example, acridine, psoralen, etc.), chelators, alkylators, and modified linkages (for example, alpha anomeric nucleic acids, etc.). The term nucleic acid molecule also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

Unless specified otherwise, the left hand end of a polynucleotide sequence written in the sense orientation is the 5′-end and the right hand end of the sequence is the 3′-end. In addition, the left hand direction of a polynucleotide sequence written in the sense orientation is referred to as the 5′-direction, while the right hand direction of the polynucleotide sequence is referred to as the 3′-direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Operably Linked:

A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Promoter:

A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

Protein or Polypeptide:

A polymer of amino acid residues, including amino acid polymers in which one or more amino acid residue is an artificial chemical mimetic of a corresponding naturally occurring amino acid, as well as to naturally occurring amino acid polymers and non-naturally occurring amino acid polymer. Multiple polymers of amino acids binding to each other are a protein complex. Protein and polypeptide may be used interchangeably throughout this application and mean at least two covalently attached amino acids, which includes proteins, polypeptides, oligopeptides and peptides. Methods of manufacturing polypeptides are known to the skilled artisan and further described herein. For example, the polypeptides disclosed herein may be produced in cell-free systems, or in prokaryotic or eukaryotic cells.

Sequence Identity/Similarity:

The primary sequence similarity between two nucleic acid molecules, or two amino acid molecules, is expressed in terms of the similarity between the sequences, otherwise referred to as sequence identity. Sequence identity is frequently measured in terms of percentage identity (or similarity or homology); the higher the percentage, the more similar are the two sequences. Methods of alignment of sequences for comparison are well known in the art.

Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations.

By way of example, the alignment tools ALIGN (Myers and Miller, CABIOS 4:11-17, 1989) or LFASTA (Pearson and Lipman, 1988) may be used to perform sequence comparisons (Internet Program© 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

Proteins orthologs are typically characterized by possession of greater than 75% sequence identity counted over the full-length alignment with the amino acid sequence of a specific reference protein, using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

When significantly less than the entire sequence is being compared for sequence identity, homologous sequences will typically possess at least 80% sequence identity over short windows of 10-20 amino acids, and may possess sequence identities of at least 85%, at least 90%, at least 95%, or at least 99%. Sequence identity over such short windows can be determined using LFASTA; methods are described at the NCSA Website; also, direct manual comparison of such sequences is a viable if somewhat tedious option.

One of skill in the art will appreciate that the sequence identity ranges provided herein are provided for guidance only; it is entirely possible that strongly significant homologs could be obtained that fall outside of the ranges provided.

The similarity/identity between two nucleic acid sequences can be determined essentially as described above for amino acid sequences. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Specifically hybridizable and specifically complementary are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Secretion Signal Sequence:

A protein sequence that can be used to direct a newly synthesized protein of interest through a cellular membrane, including the inner membrane or both inner and outer membranes of prokaryotes as well as organelle and the cell membrane of eukaryotic cells.

Split-Fluorescent Protein (SFP):

A protein complex composed of two or more protein fragments that individually are not fluorescent, but, when formed into a complex, result in a functional (that is, fluorescing) fluorescent protein complex. Split-GFP is an exemplary SFP. Individual protein fragments of a SFP are known as complementing fragments or complementary fragments. Complementing fragments which will spontaneously assemble into a functional fluorescent protein complex are known as self-complementing, self-assembling, or spontaneously-associating complementing fragments. A complemented split fluorescent protein complex is a protein complex comprising all the complementing fragments of a SFP necessary for the SFP to be active (i.e., fluorescent). Complemented fluorescent protein fluorescence is the fluorescent signal of a complemented SFP under conditions sufficient to excite the fluorescent protein. Some examples of SFP fragments include SFP tags and SFP detectors, which are further described herein.

Complementary SFP fragments are derived from the three dimensional structure of GFP, which includes eleven anti-parallel outer beta strands and one inner alpha strand. (See e.g., the GFP structure disclosed by Ormo & Remington, MMDB Id: 5742, in the Molecular Modeling Database (MMDB). The Protein Data Bank (PDB) reference is 1EMA, authors: M. Ormo & S. J. Remington, deposition: Aug. 1, 1996, class: Fluorescent Protein, title: Green Fluorescent Protein From Aequorea victoria; Ormo et al., Science, 273:1392-5, 1996; Yang et al., Nat. Biotechnol., 14:1246-51, 1996.) Typically, an SFP tag corresponds to one of the eleven beta-strands of the GFP molecule (e.g., GFP S11), and a SFP detector corresponds to the remaining strands (e.g., GFP S1-10). Other combinations of fragments are also possible, for example, as disclosed herein and in U.S. Pat. App. Pub. No. 2005/0221343 and PCT Pub. No. WO/2005/074436. Certain SFPs are further disclosed herein, including examples of Split-CFP and Split-YFP.

Split-CFP:

A SFP composed of multiple self-assembling protein fragments (e.g., a SFP detector and an SFP tag) that individually are not fluorescent, but, when complemented/assembled, form a functional (i.e., fluorescent) Cyan Fluorescent Protein (CFP). A functional (that is, fluorescing) CFP is a fluorescent protein or protein complex that can be distinguished from functional GFPs and YFPs based on excitation and emission properties. For example, a functional CFP typically has an excitation peak of approximately 430 nm wavelength and an emission peak of approximately 480 nm wavelength. For example, the functional Split-CFPs disclosed herein emit greater fluorescence at 488 nm wavelength when excited at 430 nm wavelength than the GFPs excited under the same conditions.

Examples of SFP fragments capable of forming Split-CFPs are disclosed herein. In one example, a Split-CFP detector has a consensus amino acid sequence set forth as SEQ ID NO: 22 or SEQ ID NO: 23. In some embodiments, a Split-CFP detector has an amino acid sequence set forth as SEQ ID NO: 18, SEQ ID NO: 19, SEQ ID NO: 20 or SEQ ID NO: 21.

Split-GFP:

A SFP composed of multiple self-assembling protein fragments (e.g., a SFP detector and an SFP tag) that individually are not fluorescent, but, when complemented, form a functional (i.e., fluorescent) GFP. See, e.g., U.S. Pat. App. Pub. No. 2005/0221343 and Int. Pat. App. Pub. No. WO/2005/074436; and Cabantous et al., Nat. Biotechnol., 23:102-107, 2005; Cabantous and Waldo, Nat. Methods, 3:845-854, 2006. A functional (that is, fluorescing) GFP is a fluorescent protein or protein complex that can be distinguished from functional CFPs and YFPs based on excitation and emission properties. For example, typically, a functional GFP is a fluorescent protein or protein complex with predominantly green fluorescent characteristics (e.g., an emission peak of approximately 510 nm and an excitation peak of approximately 488 nm).

In some embodiments, variations of GFP S1-10, or variations of GFP S11 may be utilized. For example, GFP S1-10 OPT (SEQ ID NO: 4) may be used as a Split-GFP S1-10 fragment. Further, for example, GFP S11214-238 (SEQ ID NO: 8), GFP S11 214-230 (SEQ ID NO: 10), GFP S11 M1 (SEQ ID NO: 12), GFP S11 M2 (SEQ ID NO: 14), GFP S11 M3 (SEQ ID NO: 16) may be used as a Split-GFP S11 fragment. Other variations are also available; see, e.g., U.S. Pat. App. Pub. No. 2005/0221343.

In other examples, Split-GFP may comprise Split-GFP fragments GFP S1-9 and GFP S10-11. GFP S1-9 corresponds to GFP beta strands 1-9 and GFP S10-11 corresponds to beta strands 10-11. Neither molecule fluoresces alone, but will form the complete fluorophore when brought into association. In some embodiments, variations of GFP S1-9, or variations of GFP S10-11 may be utilized; such variants are known, see, e.g., U.S. Pat. App. Pub. No. 2005/0221343. In other examples, a tripartite system is used that includes GFP S11, GFP S10 and GFP S1-9.

Split-YFP:

A SFP composed of multiple self-assembling protein fragments (e.g., a SFP detector and an SFP tag) that individually are not fluorescent, but, when complemented, form a functional fluorescent Yellow Fluorescent Protein (YFP). A functional (that is, fluorescing) YFP is a fluorescent protein or protein complex that can be distinguished from functional GFPs and CFPs based on excitation and emission properties. For example, a functional YFP typically has an excitation peak of approximately 515 nm and an emission peak of approximately 530 nm. For example, the functional Split-YFP molecules disclosed herein emit at least ten-fold greater fluorescence at 532 nm wavelength when excited at 510 nm wavelength than the fluorescence they emit at 510 nm wavelength when excited at 488 nm wavelength under the same conditions.

In one example, a Split-YFP detector has a consensus amino acid sequence set forth as SEQ ID NO: 30 or SEQ ID NO: 31. In some embodiments, a Split-YFP detector has an amino acid sequence set forth as SEQ ID NO: 25, SEQ ID NO: 26, SEQ ID NO: 27, SEQ ID NO: 28 or SEQ ID NO: 29.

Subcellular Compartment:

A portion or section of a cell that is less than the whole cell. For example, a subcellular compartment may be an organelle within a cell, a membrane within a cell or an area surrounding a particular structure of a cell. Examples of subcellular compartments within eukaryotic cells include cytoplasm, nucleus, mitochondria, Golgi apparatus, endoplasmic reticulum (ER), peroxisome, lysosomes, endosomes (early, intermediate, late, etc.), vacuoles, cytoskeleton, nucleoplasm, nucleolus, nuclear matrix and ribosomes. In some examples, a subcellular compartment can be defines by proximity to a particular location within a cell, for example, the post-synaptic density of a neuron. See, e.g., Alberts et al., Molecular Biology of the Cell, 5^{th} edition, New York, Garland Science, 2005.

Subcellular Localization:

The location of a molecule in relation to a subcellular compartment.

Subcellular Localization Element:

A molecule capable of directing a protein of interest to a particular subcellular compartment when the molecule is in contact with the protein. Non-limiting examples include protein, DNA, RNA, lipid, carbohydrate and small molecules capable of directing a protein to a subcellular compartment when in contact with the protein. The skilled artisan is familiar with molecules capable of directing a protein of interest to a particular subcellular compartment, and such molecules are further described herein. In some examples, the subcellular localization element is a mannose-6-phosphate moiety. In other examples, the subcellular localization element is a tag, which directs a heterologous protein that it is fused to a particular subcellular compartment. Examples of such tags are further disclosed herein.

Tag:

A polypeptide that, when fused to a heterologous protein or peptide, facilitates the detection, function, localization or isolation of the heterologous protein. Tags contemplated for use with the compositions and methods described herein include, but are not limited to, affinity tags, detection tags, SFP tags and subcellular localization elements. Although tags are often grouped into the aforementioned categories, one of skill in the art will recognize that some tags can be members of more than one group. For example, affinity tags can often be used as a detection tag, and detection tags can often be used as affinity tags. Nucleic acid encoding tags and nucleic acid constructs including nucleic acid sequences encoding tags are known to the skilled artisan and are available commercially.

An affinity tag is a polypeptide that specifically binds to (or with) an affinity reagent. For example, some affinity tags are recognized by an antibody, such as T7, FLAG, hemagglutinin (HA) VSV-G, V5 or c-myc tags. In these cases the antibody is the affinity reagent. Antibodies to these and other affinity tags are commercially available for a variety of sources. Other examples of affinity tags include affinity tags recognized by a recognized by a substrate or compound, such as a histidine tag (e.g., 6HIS; 5HIS), MBP, CBP or GST tags. In this case, the substrate or compound is the affinity reagent. Substrates to these and other affinity tags are commercially available for a variety of sources. For example, histidine tags have affinity for nickel, thus nickel is an affinity reagent for a histidine tag. In some embodiments, the nucleic acid molecules disclosed herein encode a SFP tag, such as GFP S11, GFP, S10, GFP, S1-10, or GFP S1-9. In these cases, an affinity reagent could be the corresponding SFP detector, such as GFP S1-10 or GFP S1-9.

Tagging is the process of recombinantly (or chemically) attaching a tag to a protein of interest, such as to facilitate detection or isolation of the protein.

Vector:

A nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements known in the art. An integrating vector is capable of integrating itself into a host nucleic acid. An expression vector is a vector that contains the necessary regulatory sequences to allow transcription and translation of inserted gene or genes.

### II. Overview of Several Embodiments

As disclosed herein, novel combinations of amino acid substitutions within Split-GFP result in functional Split-CFP and Split-YFP molecules. Thus, novel polypeptides comprising Split-YFP and Split-CFP molecules are provided herein. Methods of using the polypeptides described herein are also disclosed. Non-limiting examples of methods of using these SFPs include methods of determining the subcellular localization of a protein and methods of determining the membrane topology of a protein.

Polypeptides comprising SFP detectors are provided. In some embodiments, the polypeptides comprise a Split Fluorescent Protein (SFP) detector comprising an amino acid sequence set forth as SEQ ID NO: 22, wherein the SFP detector complements with a SFP tag to form a functional Split-Cyan Fluorescent Protein. For example, a polypeptide comprising a Split Fluorescent Protein (SFP) detector comprising an amino acid sequence set forth as SEQ ID NO: 23, wherein the SFP detector complements with a SFP tag to form a functional Split-Cyan Fluorescent Protein. For example, a polypeptide comprising an amino acid sequence set forth as SEQ ID NO: 18, SEQ ID NO: 19, SEQ ID NO: 20 or SEQ ID NO: 21, wherein the polypeptide complements with a SFP tag to form a functional Split-Cyan Fluorescent Protein.

In some embodiments, the polypeptides comprise a Split Fluorescent Protein (SFP) detector comprising an amino acid sequence set forth as SEQ ID NO: 30, wherein the SFP detector complements with a SFP tag to form a functional Split-Yellow Fluorescent Protein. For example, a Split Fluorescent Protein (SFP) detector comprising an amino acid sequence set forth as SEQ ID NO: 31, wherein the SFP detector complements with a SFP tag to form a functional Split-Yellow Fluorescent Protein. For example, a polypeptide comprising an amino acid sequence set forth as SEQ ID NO: 25, SEQ ID NO: 26, SEQ ID NO: 27, SEQ ID NO: 28 or SEQ ID NO: 29.

In some embodiments, the polypeptides disclosed herein are fused to a subcellular localization element.

Some embodiments include a nucleic acid molecule comprising a nucleotide sequence encoding a polypeptide described herein. For example, a nucleic acid molecule comprising a nucleotide sequence encoding a polypeptide comprising an amino acid sequence set forth as any one SEQ ID NO: 18, SEQ ID NO: 19, SEQ ID NO: 20 or SEQ ID NO: 21, SEQ ID NO: 21, SEQ ID NO: 22, SEQ ID NO: 25, SEQ ID NO: 26, SEQ ID NO: 27, SEQ ID NO: 28, SEQ ID NO: 29, SEQ ID NO: 30 or SEQ ID NO: 31.

In some embodiments a host cell comprising a nucleic acid molecule as described herein is provided. For example, a host cell comprising a nucleic acid molecule comprising a nucleotide sequence encoding a polypeptide comprising an amino acid sequence set forth as any one SEQ ID NO: 18, SEQ ID NO: 19, SEQ ID NO: 20 or SEQ ID NO: 21, SEQ ID NO: 21, SEQ ID NO: 22, SEQ ID NO: 25, SEQ ID NO: 26, SEQ ID NO: 27, SEQ ID NO: 28, SEQ ID NO: 29, SEQ ID NO: 30 or SEQ ID NO: 31.

Methods of determining the subcellular localization of a protein are provided. In some embodiments, the methods include providing within at least one host cell a first polypeptide comprising a first subcellular localization element and a first Split Fluorescent Protein (SFP) detector comprising a polypeptide comprising an amino acid sequence set forth as SEQ ID NO: 23 or SEQ ID NO: 31, wherein the first subcellular localization element localizes the first polypeptide to a first subcellular compartment; providing within the host cell a second polypeptide comprising a test protein fused to a SFP tag, and detecting fluorescence of the first SFP detector complemented with the SFP tag in the host cell, wherein the presence of fluorescence of the first SFP detector complemented with the SFP tag identifies the test protein as localized to the first subcellular compartment, thereby determining a subcellular localization of a protein.

In some embodiments of the methods of determining the subcellular localization of a protein, the method further comprises the test protein is a membrane protein, the SFP tag is fused to the N- or C-terminus of the test protein and the presence of fluorescence of the first SFP detector complemented with the SFP tag in the host cell further identifies the terminus of the test protein fused to the SFP tag as on the same side of the membrane as the first SFP detector.

In some embodiments of the methods of determining the subcellular localization of a protein, providing the first polypeptide or the second polypeptide within the host cell comprises expressing the first or second polypeptide within the host cell, contacting the host cell with the first or second polypeptide, or a combination thereof.

In some embodiments of the methods of determining the subcellular localization of a protein, the method further comprises providing within the host cell a third polypeptide comprising a second subcellular localization element and a second SFP detector, wherein the second subcellular localization element localizes the third polypeptide to a second subcellular compartment, and wherein the second SFP detector can be differentially detected from the first SFP detector when complemented with the SFP tag, and detecting fluorescence of the second SFP detector complemented with the SFP tag in the host cell, wherein the presence of fluorescence of the second SFP detector complemented with the SFP tag identifies the test protein as localized to the second subcellular compartment. In some such embodiments, the first and third polypeptides comprise any two polypeptides selected from the group consisting of a polypeptide comprising an amino acid sequence set forth as SEQ ID NO: 23, a polypeptide comprising an amino acid sequence set forth as SEQ ID NO: 31 and a polypeptide comprising a Split-GFP SFP detector. In some such embodiments, the test protein is a membrane protein, the SFP tag is fused to the N- or C-terminus of the test protein, the presence of fluorescence of the first SFP detector complemented with the SFP tag in the host cell identifies the terminus of the test protein fused to the SFP tag as on the same side of the membrane as the first SFP detector, and the presence of fluorescence of the second SFP detector complemented with the SFP tag in the host cell identifies the terminus of the test protein fused to the SFP tag as on the same side of the membrane as the second SFP detector. In some such embodiments, providing the first polypeptide, the second polypeptide or the third polypeptide within the host cell comprises expressing the first, second or third polypeptide within the host cell, contacting the host cell with the first, second or third polypeptide, or a combination thereof.

In some embodiments, a method for detecting the localization of a test protein to one or more of a plurality of subcellular components in a cell is provided. For example, such methods include providing within the cell a polypeptide comprising the test protein and a SFP tag, providing within the cell a plurality of SFP detectors complementary to the SFP tag at least one of which is a polypeptide comprising an amino acid sequence set forth as SEQ ID NO: 23 or SEQ ID NO: 31, wherein each of the SFP detectors is capable of producing different color fluorescence upon complementation with the SFP tag and each of the SFP detectors is fused to a subcellular localization element that localizes the SFP detector to a different subcellular compartment, and detecting the various color fluorescence signals in cell, thereby detecting the localization of the test protein to one or more of the subcellular compartments. In some such embodiments, the plurality of SFP detectors comprises a polypeptide comprising an amino acid sequence set forth as SEQ ID NO: 23, a polypeptide comprising an amino acid sequence set forth as SEQ ID NO: 31, a Split-GFP SFP detector or a combination of two or more thereof. In some such embodiments, providing the polypeptide comprising the test protein and the SFP tag or the plurality of SFP detectors within the host cell comprises expressing the polypeptide comprising the test protein and the SFP tag or the plurality of SFP detectors within the host cell; contacting the host cell with the polypeptide comprising test protein and the SFP tag or the plurality of SFP detectors or a combination thereof.

Methods of determining the membrane topology of a membrane protein are provided. In some embodiments, such methods include providing within at least one host cell a first polypeptide comprising a first subcellular localization element and a first Split Fluorescent Protein (SFP) detector comprising a polypeptide comprising an amino acid sequence set forth as SEQ ID NO: 23 or SEQ ID NO: 31, wherein the first subcellular localization element localizes the first polypeptide to one side of a membrane of the host cell, providing within the host cell a second polypeptide comprising a test membrane protein, the N- or C-terminus of which is fused to a SFP tag, and detecting fluorescence of the first SFP detector complemented with the SFP tag in the host cell, wherein the presence of fluorescence of the first SFP detector complemented with the SFP tag in the host cell identifies the membrane orientation of the terminus of test protein fused to the SFP tag as on the same side of the membrane as the first SFP detector, thereby determining the topology of a membrane protein. In some such embodiments, providing the first polypeptide or the second polypeptide within the host cell comprises expressing the first or second polypeptide within the host cell, contacting the host cell with the first or second polypeptide, or a combination thereof.

In some embodiments of a method of determining the membrane topology of a membrane protein, the method further comprises providing within the host cell a third polypeptide comprising a second subcellular localization element and a second Split Fluorescent Protein (SFP) detector, wherein the second subcellular localization element localizes the third polypeptide to the opposite side of membrane of the host cell compared to the first subcellular localization element, and wherein the second SFP detector polypeptide can be differentially detected from the first SFP detector when complemented with the SFP tag, and detecting fluorescence of the second SFP detector complemented with the SFP tag in the host cell, wherein the presence of fluorescence of the second SFP detector complemented with the SFP tag in the host cell identifies the membrane orientation of the terminus of test protein fused to the SFP tag as on the same side of the membrane as the second SFP detector. In some such embodiments, the first and third polypeptides comprise any two polypeptides selected from the group consisting of a polypeptide comprising an amino acid sequence set forth as SEQ ID NO: 23, a polypeptide comprising an amino acid sequence set forth as SEQ ID NO: 31 and a polypeptide comprising a Split-GFP SFP detector. In some such embodiments, providing the first polypeptide, the second polypeptide or the third polypeptide within the host cell comprises expressing the first, second or third polypeptide within the host cell, contacting the host cell with the first, second or third polypeptide, or a combination thereof.

In some embodiments, the host cell or cell is a eukaryotic cell. In some embodiments, detecting SFP fluorescence in the host cell or cell comprises flow cytometry. In some embodiments, the host cell or cell that expresses the test protein is selected.

Some embodiments provide a kit, comprising a nucleic acid construct comprising a nucleic acid molecule encoding a polypeptide comprising an amino acid sequence set forth as SEQ ID NO: 23 or SEQ ID NO: 31 and a multiple cloning site adjacent thereto, such that an encoding sequence inserted into the multiple cloning site results in a nucleic acid molecule that encodes a protein encoded by the encoding sequence fused with the protein encoded by the nucleic acid molecule and instructions for use thereof.

### III. SPFs

SFPs are a protein complex composed of two or more protein fragments that individually are not fluorescent, but, when formed into a complex, result in a functional (that is, fluorescing) fluorescent molecule. Complementary sets of such fragments are also known as a SFP system. Split-YFPs, Split-GFPs and Split-CFPs are disclosed herein. Also disclosed are nucleic acid molecules The SFPs disclosed herein are self-complementing SFPs. The embodiments described herein utilize SFP tags and SFP detectors, which are based on a complementary set of SFP fragments. An SFP tag is a SFP fragment that, when fused to a heterologous protein or peptide (i.e., a test protein), allows detection of the heterologous protein using the complementary SFP fragment. The SFP detector is the SFP fragment corresponding to the SFP tag. Thus, an SFP tag and the complementary SFP detector are two complementing fragments of a SFP.

In the context of a SFP composed of two complementary fragments, wherein the SFP has an 11 beta-strand barrel structure similar to GFP, the SFP tag typically will comprise one or two strands of the 11 beta-strand barrel structure and the SFP detector typically will comprise the remaining strands of the 11 beta-strand barrel structure. Typically, when fused to a test protein, a SFP tag is substantially non-perturbing to the structure of the test protein. Small, engineered SFP tags can be engineered to be less perturbing to fusion protein folding and solubility relative to the same proteins fused to the full-length fluorescent protein (see, e.g., Cabantous et al., Nat. Biotechnol., 23:102-107, 2005; Pedelacq et al., Nat. Biotechnol., 24:79-88, 2006). For example, GFP S11 may be an SFP tag, in which case GFP S1-10 would be the complementary SFP detector. In some examples, the SFP tag and SFP detector are based on a circular permutant of a SFP, for example as described herein and in U.S. Pat. App. Pub. No. 2005/0221343 and PCT Pub. No. WO/2005/074436.

Construction of a test protein fused to a SFP tag or SFP detector is typically accomplished via cloning of the nucleic acid encoding the test protein into a nucleic acid construct encoding the SFP tag or SFP detector. SFPs, SFP systems, a number of specifically engineered tag and detector fragments of a SFP, as well as DNA constructs and vectors use thereof are disclosed herein and known to the skilled artisan. See, e.g., U.S. Pat. App. Pub. No. 2005/0221343; Int. Pat. App. Pub. No. WO/2005/074436; Cabantous et al., Nat. Biotechnol., 23:102-107, 2005; Cabantous and Waldo, Nat. Methods, 3:845-854, 2006.) Typically, the SFPs include two SFP fragments, such as a SFP tag (typically corresponding to GFP S11) and a SFP detector (typically corresponding to GFP S1-10). Other SFPs are disclosed herein.

Polypeptides comprising Split-GFP fragments are known to the skilled artisan and further described herein. See, e.g., U.S. Pat. App. Pub. No. 2005/0221343 and Int. Pat. App. Pub. No. WO/2005/074436, and Cabantous et al., Nat. Biotechnol., 23:102-107, 2005; Cabantous and Waldo, Nat. Methods, 3:845-854, 2006. For example, in some embodiments, GFP S1-10 OPT (SEQ ID NO: 4) may be used as a Split-GFP S1-10 fragment. A corresponding SFP tag, for example, GFP S11 M3 (SEQ ID NO: 16) may be used as the complementing Split-GFP S11 fragment. Other variations are also available; see, e.g., U.S. Pat. App. Pub. No. 2005/0221343. The polypeptides comprising complementing Split-GFP fragments disclosed herein will form a functional GFP molecule when complemented.

Disclosed herein are polypeptides comprising fragments of Split-CFP molecules, including Split-CFP detectors. In some embodiments, a Split-CFP detector includes a consensus amino acid sequence set forth as:

MSKGEELFTGVVPILX₁[16]ELX₂[19]GX₃[21]VNGHKFSVRGEGEGDATIGKLTL KFICTTGKLPVPWPTLVTTLTW[66]GVQCFSRYPDHMKRHDFFKSAMPEGYVQ ERTIX₄[99]FKDDGKYKTRAVVKFEGDTLVNRIX₅[124]LKGTDFKEDGNILGHKL EYNFNSD[148]NVYITADKQKNGIKANFTX₆[167]RHNVEDGSVQLADHYQQNT PIGDGPVLLPDNHYLSTQSVLSKDPNEKGS (SEQ ID NO: 22), wherein X₁ is V or I, X₂ is D or E, X₃ is D, E or N, X₄ is S or T, X₅ is E or V, and X₆ is V or I. The disclosure also provides sequences having at least 80%, at least 85%, at least 90%, at least 95%, or at least 98% sequence identity to SEQ ID NO: 22, wherein residue 16 is V or I, residue 21 is D or E, residue 21 is D, E or N, residue 66 is W, residue 99 is S or T, residue 124 is E or V, residue 148 is D, residue 167 is V or I and residue 205 is S. In other examples, a Split-CFP detector includes a consensus amino acid sequence set forth as
MSKGEELFTGVVPILVELEGEVNGHKFSVRGEGEGDATIGKLTLKFICTTGKLP VPWPTLVTTLTWGVQCFSRYPDHMKRHDFFKSAMPEGYVQERTISFKDDGKY KTRAVVKFEGDTLVNRIX₁[124]LKGTDFKEDGNILGHKLEYNFNSDNVYITAD KQKNGIKANFTX₂[167]RHNVEDGSVQLADHYQQNTPIGDGPVLLPDNHYLSTQ S[205]VLSKDPNEKGS (SEQ ID NO: 23), wherein X₁ is E or V, and X₂ is V or I. The disclosure also provides sequences having at least 80%, at least 85%, at least 90%, at least 95%, or at least 98% sequence identity to SEQ ID NO: 23, wherein residues 19 and 21 are E, residue 66 is W, residue 124 is E or V, residue 148 is D, residue 167 is V or I and residue 205 is S. Specific examples of amino acid sequence comprising a Split-CFP detector include the amino acid sequences set forth as SEQ ID NO: 18, SEQ ID NO: 19, SEQ ID NO: 20, or SEQ ID NO: 21.

Also disclosed herein are polypeptides comprising fragments of Split-YFP molecules, including Split-YFP detectors. In some embodiments, a Split-YFP detector includes a consensus amino acid sequence set forth as:

MSKGEELFX₁[9]GVVPILVELDGDVNGHKFSVRGEGEGDATIGKLTLKFICTTGK LPVPWPTLVTTLX₂[65]YGVQX₃[70]FSRYPDHMKX₄[80]HDFFKSAMPEGYVQE RTIX₅[99]FKDDGKYKTRAVVKFEGDTLVNRIELKGTDFKEDGNILGHKLEYNF NSHNVYITADKQKNGIKANFTVRHNVEDGSX₆[176]QLADHYQQNTPIGDGX₇[192]VLLPDNHX₈[200]LSY[203]X₉[204]X₁₀[205]VLSKX₁₁[210]PNEKGS (SEQ ID NO: 30), wherein X₁ is T or N, X₂ is T, L, G or A, X₃ is C or S, X₄ is R or K, X₅ is S or F, and X₆ is V or I, X₇ is P or H, X₈ is Y or F, X₉ is Q, H or E, X₁₀ is T, S or A and X₁₁ is D or V. The disclosure also provides sequences having at least 80%, at least 85%, at least 90%, at least 95%, or at least 98% sequence identity to SEQ ID NO: 30, wherein residue 9 is T or N, residue 65 is T, L, G or A, residue 70 is C or S, residue 80 is R or K, residue 99 is S or F, and residue 176 is V or I, residue 192 is P or H, residue 200 is Y or F, residue 203 is Y, residue 204 is Q, H or E, residue 205 is T, S or A and residue 210 is D or V. In other examples, a Split-YFP detector includes a consensus amino acid sequence set forth as
MSKGEELFTGVVPILVELDGDVNGHKFSVRGEGEGDATIGKLTLKFICTTGKLP VPWPTLVTTLX₁[65]YGVQCFSRYPDHMKRHDFFKSAMPEGYVQERTISFKDDG KYKTRAVVKFEGDTLVNRIELKGTDFKEDGNILGHKLEYNFNSHNVYITADKQ KNGIKANFTVRHNVEDGSVQLADHYQQNTPIGDGPVLLPDNHYLSY[203]QX₂[205]VLSKDPNEKGS (SEQ ID NO: 31), wherein X₁ is T, L, G or A and X₂ is S, or X₁ is T and X₂ is S or A. The disclosure also provides sequences having at least 80%, at least 85%, at least 90%, at least 95%, or at least 98% sequence identity to SEQ ID NO: 31, wherein residue 65 is T, L, G or A and residue 203 is Y, and residue 205 is S. Specific examples of amino acid sequence comprising a Split-YFP detector include the amino acid sequences set forth as SEQ ID NO: 25, SEQ ID NO: 26, SEQ ID NO: 27, SEQ ID NO: 28, or SEQ ID NO: 29.

In a particular example, a SFP detector (for example, a Split-CFP, Split-YFP) is disclosed which has at least 80%, at least 90%, at least 95%, at least 98%, such as 80%, 82%, 85%, 90%, 93%, 95%, 98% or 100% sequence identity with an amino acid sequence set forth by any one of SEQ ID NO: 18, SEQ ID NO: 19, SEQ ID NO: 20, or SEQ ID NO: 21, SEQ ID NO: 22, SEQ ID NO: 23, SEQ ID NO: 25, SEQ ID NO: 26, SEQ ID NO: 27, SEQ ID NO: 28, or SEQ ID NO: 29, SEQ ID NO: 30 or SEQ ID NO: 31, wherein the SFP detector retains the ability to complement with a SFP tag to form a functional fluorescence protein (e.g., CFP or YFP).

The SFP detectors disclosed herein are capable of complementing with a corresponding SFP tag to form a function fluorescent protein. For example, the Split-CFP detectors disclosed herein complement with a SFP tag to form a functional CFP and the Split-YFP detectors disclosed herein complement with a SFP tag to form a functional YFP.

In some examples, the polypeptides comprising SFP detectors may be fused to a subcellular localization element as described herein. The skilled artisan is familiar with methods of generating a polypeptide comprising a SFP detector fused to a subcellular localization element. In some examples, the subcellular localization element is fused to the N-terminus, the C-terminus or an internal portion of the polypeptide.

In some examples, the SFP detector is fused to another protein of interest. The polypeptides included herein may vary in length according to the specific application. For example, in some embodiments, the polypeptides are about at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, more, fewer or an in between number of amino acids in length, wherein the polypeptide comprises a SFP detector as described herein and wherein the SFP detector retains the ability to complement with a SFP tag to form a functional fluorescence protein (e.g., CFP or YFP).

In some examples, the polypeptides and the nucleic acid molecules disclosed herein are isolated polypeptides or isolated nucleic acid molecules.

The polypeptides comprising the Split-SFP detectors described herein (e.g., Split-YFP, Split-CFP and Split-GFP detectors) are useful in numerous methods, assays, systems, kits, etc. described herein and known to the skilled artisan, for example, as described in, e.g., U.S. Pat. App. Pub. No. 2005/0221343, PCT Pub. No. WO/2005/074436, U.S. Pat. No. 7,666,606; and U.S. Pat. No. 7,585,636, each of which is incorporated herein in its entirety.

### IV. Determining Subcellular Localization

Provided herein are methods of detecting, differentiating and monitoring the subcellular location of one or more proteins in cells, including living, fixed and unfixed cells, detecting proteins that interact in defined subcellular compartments, tracking the transport of proteins through and out of the cell, identifying cell surface expression, monitoring and quantifying protein secretion, and screening for mediators of localization, transport and/or secretion of proteins. These assays may also be scaled to high-throughput screening of protein variants with modified subcellular localization characteristics.

For example, in one embodiment, a test protein or group of test proteins may be screened for localization to a particular subcellular compartment, including without limitation the nucleus, cytoplasm, plasma membrane, endoplasmic reticulum, Golgi apparatus, filaments such as actin and tubulin filaments, endosomes, peroxisomes and mitochondria. Briefly, a polynucleotide construct encoding a fusion of the test protein and a SFP tag is expressed in cells containing a SFP detector complementary to the SFP tag. The complementary SFP detector comprises or is operably linked to a subcellular localization element capable of directing the SFP detector to the desired subcellular compartment. In some examples, where cytoplasm expression of the test protein is to be assayed, the subcellular localization element allows the SFP detector to be localized in the cytosol. The SFP detector may be expressed in the cell or transfected into the cell; such methods are known to the skilled artisan and further described herein.

The expressed test protein-SFP tag fusion will only be able to complement with the assay fragment if it is able to gain access to the same subcellular compartment the assay fragment has been localized to. Thus, for example, if the test protein comprises a mitochondrial localization signal, a fusion of the test protein with a SFP tag would be localized to the mitochondria. A SFP detector localized to the mitochondria will be available to complement with the SFP tag and generate fluorescence in mitochondria, which can then be detected according to standard methods known to the skilled artisan and as described herein. The method may be used to identify proteins that localize to a particular subcellular compartment or structure and to identify novel localization signals. In another illustrative embodiment, a test protein known to localize to the nucleus is generated as a fusion protein with a SFP tag. A complementary SFP detector is operably linked to a subcellular localization element that directs the SFP detector to the nucleolus. Expressing the test protein-SFP tag fusion in a cell or otherwise providing it to a cell containing the nuclear-localized SFP detector brings the two complementary fragments into proximity resulting in complementation and formation of a fluorescent molecule that can be detected according to standard methods known to the skilled artisan and as described herein. The method may be used to screen for agents that interfere with the localization of the test protein to a particular subcellular compartment.

In some applications, the test protein-SFP tag fusion may also be designed to co-localized with the SFP detector fragment (for complementation to occur), for examples, in methods where the effect of a drug on subcellular localization of the test protein localization is being evaluated. In such methods a decrease or increase in the fluorescent emission of the complimented SFP in response to the drug indicates an effect of the drug on the localization of the test protein.

In some embodiments of detecting subcellular localization of a test protein to a cellular compartment, expression of the test protein either precedes or follows the expression or transfection of the SFP detector, in order to eliminate non-specific fluorescence resulting from transient co-localization of the SFP tag and detector in the course of processing or transport to a particular subcellular compartment. In some applications, it may be desirable to visualize protein transport through the cell over a time course, and in such applications, the test protein-SFP tag and SFP detector fragments may be co-expressed, from one or more constructs, and optionally under the control of individually inducible promoter systems.

Thus, in one embodiment, the SFP detector fused to a subcellular localization element is pre-localized to the compartment of interest. This may be achieved by inducing the expression of a polynucleotide encoding the SFP detector fused to the subcellular localization element, terminating induction, and then inducing expression of the test protein-SFP tag fusion protein through a separately inducible system. Complementation of the pre-localized SFP detector fragment and the expressed test protein-SFP tag fusion results in fluorescence in the specialized cell compartment, which can be detected according to known methods and as described herein.

In a related embodiment, the cells used to conduct the method express or are provided with plurality of complementary SFP detectors, each of which is localized to a different subcellular compartment (e.g., by fusion with different subcellular localization elements that confer localization to different subcellular compartments) and designed or selected to produce different color fluorescence upon complementation with the SFP tag. For example, the plurality of SFP detectors may contain a GFP S1-10 SFP detector, a CFP S1-10 SFP detector and/or a YFP S1-10 SFP detector, each of which is fused to a subcellular localization element that localizes the detector to a different subcellular compartment. Thus, the color of the fluorescence generated when self-complementation occurs correlates with and localizes to a particular subcellular compartment or structure. Such an assay may be used to screen proteins for their subcellular localization profiles at fixed time points or in real time and to visualize protein trafficking dynamically.

For example, to visualize a test protein's transport and localization from the ER to the Golgi, two SFP detectors are used, one fused to an ER-targeted subcellular localization element and selected to produce cyan fluorescence upon complementation with a SFP tag present in the ER, and the other fused to a Golgi-targeted subcellular localization element and selected to produce yellow fluorescence upon complementation with the SFP tag present in the Golgi. Optionally, a third assay fragment, for example, may be fused to a endosome-targeted subcellular localization element and selected to produce green fluorescence upon complementation with a SFP tag located in endosomes. Optionally, a fourth assay fragment selected to produce red fluorescence could be added to the extracellular media, in excess, in order to capture any SFP tag that is secreted by the cell. A test protein can be fused to the SFP tag; thereby allowing detection of the subcellular localization of the test-protein-SFP tag fusion. Thus, this illustrative combination of fragments and colors could be used to monitor the secretion pathway of a test protein.

Similarly, the secretion assay illustrated above may be used to screen for agents that inhibit or otherwise modulate protein secretion, by adding agent(s) to the cells and observing changes in trafficking and/or secretion yields. Thus, for example, an SFP detector may be targeted to the Golgi to evaluate changes to the secretion pathway of a test protein-SFP tag fusion in the presence of a test agent (e.g., a drug). If a test protein is destined for secretion or export, then complementation between the test protein-SFP tag fusion and the SFP detector will occur in the Golgi, and Golgi vesicles would be detected using the complemented SFP fluorescence. Conversely, the absence of complemented SFP fluorescence indicates that the test protein's secretion pathway is altered by the drug.

In a related embodiment, the secretion assay described above enables the quantification of secreted protein yield, by comparing the fluorescence observed in the extracellular environment (e.g., growth media) with a calibration curve obtained with a soluble control protein and the same “extracellular” SFP detector. In one embodiment of a protein secretion quantitative assay, the test protein is expressed in fusion with the SFP tag (e.g., GFP S11) for a time sufficient to permit secretion of the test protein-SFP tag fusion if secreted. Cells are then pelleted from growth media and an excess of a complementary SFP detector is added to the supernatant. Fluorescence is then measured and used to determine secreted protein quantity.

Secreted proteins identified as above may also be purified by including a modification to the SFP detector or tag that can be used as an affinity tag. Typically, this will comprise a sequence of amino acid residues that functionalize the SFP fragment to bind to a substrate that can be isolated using standard purification technologies. In one embodiment, a SFP fragment is functionalized to bind to glass beads, using chemistries well known and commercially available (e.g., Molecular Probes Inc.). Alternatively, the SFP fragment may be modified to incorporate histidine residues in order to functionalize the SFP fragment to bind to metal affinity resin beads. In a specific embodiment, a GFP S11 tag fragment, engineered so that all outside pointing residues in the β-strand are replaced with histidine residues, is used (see, e.g., U.S. application Ser. No. 10/973,693). This HIS-tag fragment is non-perturbing to test proteins fused therewith, and is capable of complementing with a SFP detector and forming a functional SFP. The HIS-tag fragment can be used to purify secreted proteins from growth media using standard purification techniques.

In some embodiments, the methods may be used to determine the cell surface expression of a protein. Test protein-SFP tag fusions are expressed in the cell. A complementary SFP detector is added to the surface of the cells (e.g., by adding to the growth media). If the test protein-SFP tag fusion is expressed on the cell surface, complementation with the SFP detector occurs at the cell surface, and complemented SFP fluorescence can be detected at the cell surface according to known methods.

The methods described herein, including methods of determining the subcellular localization of a protein involving the use of multiple SFP detectors that can be differentially detected may be combined with flow cytometry to detect cells displaying a particular fluorescence. For example, if a library of test proteins is being screened for localization to a particular subcellular compartment (e.g., the nucleus or the mitochondria), multiple SFP detectors that can be differentially detected are fused to appropriate subcellular localization elements for targeting to particular subcellular compartments. This will permit flow cytometry detection of cells expressing test protein-SFP tag fusions that localize to a particular compartment. Further, by using FACS techniques, cells expressing a particular test-protein (as identified by detection of a particular SFP fluorescence) can be sorted and isolated.

Yet another aspect of the invention relates to assays used to screen for agents that modulate protein localization. In one embodiment, a test protein-SFP tag fusion is transfected into a cell, and an agent (e.g., a drug) of interest is added to the cell. Complementary SFP detector fused to different subcellular localization elements (to direct the SFP detector to different subcellular compartments), resulting in different fluorescent colors upon complementation of the SFP detector and SFP tag, depending on the localization of the test protein-SFP tag fusion. The assay fragments are expressed in or transfected into the cell following the addition of the drug. Detection of complemented SFP fluorescence in the host cell is used to identify the subcellular compartment that the protein-SFP tag fusion is localized to. A change in fluorescence emission in response to the agent indicates that the agent induces altered subcellular localization of the protein-SFP-tag fusion.

The methods described herein are easily extended to methods involving libraries of test proteins, for example a library of variants of a particular protein. The skilled artisan is familiar with protein libraries, and such libraries, as well as methods of making them are further described herein. The disclosed methods involving use of libraries of test proteins include at least two host cells, each expressing a different member of the library of test proteins.

Detection of SFP fluorescence in the embodiments described herein is accomplished according to standard methods of detecting fluorescent proteins. The SFP is exposed to an appropriate excitation wavelength, and light emitted at the corresponding emission wavelength is detected. Such methods are well known the skilled artisan, and systems for detecting fluorescent proteins are commercially available. For example, Flow cytometry methods and/or fluorescence microscopy, such as confocal microscopy methods may be used.

### V. Determining Membrane Topology of Membrane Proteins

Provided herein are methods of determining the membrane topology of a membrane protein. The methods utilize the Split-fluorescent proteins described herein. For example, the methods of determining the subcellular localization of a protein described herein may be adapted for determination of membrane topology of a membrane protein if the test protein is a membrane protein. For example, a SFP tag can be fused to a test membrane protein (N-terminus, C-terminus, or internally), and the fusion protein expressed within a cell or subcellular compartment. The protein becomes embedded or anchored within a target membrane. For illustration, assume that the membrane has an internally-facing side (to the interior of the cell compartment) and an external side (to the exterior of the cell compartment). An SFP detector complementing the SFP tag is expressed or added using a protein transfection reagent, and is directed to the interior side of the membrane using a subcellular localization element, for example. If the test protein is oriented with the SFP tag directed to the interior of the membrane, complementation occurs and fluorescence is detectable. If the SFP tag is oriented to the exterior of the compartment, complementation does not occur and no or reduced SFP fluorescence is detectable. Simultaneous detection of more than one possible localization event can be performed using multiple SFP detectors that can be differentially detected. For example, a YFP S1-10 SFP detector (as described herein) can be directed to the outside of the membrane, using a subcellular localization element, for example, and a GFP S1-10 or CFP S1-10 SFP detector is directed to the interior, using a subcellular localization element, for example. Detection of Split-YFP fluorescence in the cell indicates the tag is localized to the exterior of the membrane, while detection of Split-GFP or Split-CFP fluorescence indicates that the tag is localization to the interior of the membrane. Any combination of SFP detectors that may be differentially detected may be used. The order of expression of the tagged protein and assay fragments can be reversed if desired to increase signal-to-noise and improve specificity. For example, the assay fragment(s) could be transiently-expressed, followed by the tagged test protein.

### VI. Selecting a Host Cell Expressing a Test Protein

Some embodiments further include selecting a host cell expressing a test protein. For example, a test protein for which the subcellular localization has been identified using the methods described herein. For example, some embodiments include selecting the host cell comprising a test protein expressed from a nucleic acid within the host cell, so that the nucleic acid may be isolated from the host cell. As used herein, selecting a host cell includes selecting a particular host cell, as well as selecting a number of cells (e.g., a colony of host cells) comprising the host cell. Selecting a host cell comprising nucleic acid encoding a test protein involves identifying the host cell that expresses the test protein, and selecting the identified host cell.

Methods of selecting a host cell are well known to the skilled artisan and are described herein. In some embodiments, selecting the host cell comprises manual selection of the host cell, for example, by picking a colony comprising the host cell using a sterile toothpick. In some embodiments, selecting the host cell comprises robotic selection of the host cell, for example by a colony picking robot. Such robots and methods of using such robots are known to the skilled artisan; also such robots are available commercially, for example from Norgren Systems (No. CP 700; Ronceverte, W. Va.) and BioRad (VersArray, No. 2856; Hercules, Calif.). In some embodiments the selected host cell is cultured for further study.

In some embodiments, selecting a host cell comprising nucleic acid encoding a test protein involves identifying the host cell corresponding to the detected SFP fluorescence used to identify the subcellular localization of the test protein, and selecting the identified host cell. Methods of identifying a host cell corresponding to particular SFP fluorescence are known to the skilled artisan and are further described herein. For example, flow cytometry and FACS techniques may be used to identify and select host cells comprising particular SFP fluorescence, for example SFP fluorescence produced by a Split-CFP, Split-GFP or Split-YFP molecule.

### VII. Subcellular Localization Elements

Various subcellular localization elements are known to the skilled artisan and commercially available. These subcellular elements are used to direct proteins (e.g., Split-fluorescent protein fragments) to particular cellular subcellular locations. Subcellular localization elements capable of targeting proteins to at least the nucleus, cytoplasm, plasma membrane, endoplasmic reticulum, Golgi apparatus, actin and tubulin filaments, endosomes, peroxisomes, mitochondria and outside the cell of eukaryotic cells are known. Subcellular localization elements capable of directing proteins to subcellular compartments of prokaryotic cells (e.g., cytoplasm, cytoplasmic membrane, cell wall and outside the cell) are also known and are familiar to the skilled artisan.

In some examples, subcellular localization elements require a specific orientation (e.g., N- or C-terminal) relative to the protein to which the element is attached. For example, the nuclear localization signal (NLS) of the simian virus 40 large T-antigen must be oriented at the C-terminus of a protein to direct that protein to the nucleus. Thus, in examples where the test protein-SFP tag fusion is to be localized to the nucleus, a NLS could be fused to the C-terminus of the test-protein-SFP tag fusion. Similarly, in examples where and SFP detector is to be targeted to the nucleus, a NLS could be fused to the C-terminus of the SFP detector.

In some embodiments, a mannose-6-phosphate tag is used as a subcellular localization element. For example, the mannose-6 phosphate tag can be added to a test protein or a SFP detector prior to provision of the test protein or SFP detector to a host cell in embodiments of identifying a subcellular localization of a test protein described herein. Methods of fusing a protein with the mannose-6-phosphate tag are known to the skilled artisan.

Table 1 provides examples of subcellular localization elements capable of directing proteins to the nuclear, Golgi, mitochondrial, and ER compartments of eukaryotic cells, together with orientation information. Table 2 provides the protein and exemplary nucleic acid sequences of several subcellular localization elements. Other localization signal sequences are known to the skilled artisan, are commercially available and may be used with the embodiments described herein.

TABLE 1
Examples of eukaryotic subcellular localization elements

| | | Position in fusion | | |
|---|---|---|---|---|
| Localization tag | Localization signal | protein | Function | References |
| Nucleus | Nuclear localization | C-terminus | For localized | Kalderon et al., Cell, |
| | signal (NLS) of the | | expression in the | 39: 499-509, 1984; |
| | simian virus40 large | | nucleus of | Lanford, et al., Cell, |
| | T-antigen | | mammalian cells. | 46: 575-582, 1986 |
| Golgi | Sequence encoding | N-terminus | This region of human | Watzele and Berger, |
| | the N-terminal 81 | | beta 1,4-GT contains | Nucleic Acids Res., |
| | amino acids of | | the membrane- | 18: 7174, 1990; |
| | human beta 1,4- | | anchoring signal | Yamaguchi and |
| | galactosyltransferase | | peptide that targets | Fukuda, J. Biol. |
| | (GT) | | the fusion protein to | Chem., 270: 12170-12176, |
| | | | the trans-medial | 1995; Llopis et |
| | | | region of the Golgi | al., Proc. Natl. Acad. |
| | | | apparatus | Sci. U.S.A., 95: 6803-6808, |
| | | | | 1998 |
| Mitochondria | Mitochondrial | N-terminus | Designed for labeling | Rizzuto et al., J. Biol. |
| | targeting sequence | | of mitochondria | Chem., 264: 10595-10600, |
| | derived from the | | | 1989; Rizzuto |
| | precursor of subunit | | | et al., Curr. Biol., |
| | VIII of human | | | 5: 635-642, 1995 |
| | cytochrome C | | | |
| | oxidase | | | |
| Endoplasmic | (ER) targeting | N-terminus | For labeling of the | Munro and Pelham, |
| reticulum (ER) | sequence of | | endoplasmic | Cell, 48: 899-907, |
| | calreticulin | | reticulum in | 1987; Fliegel et al., J. |
| | | | mammalian cells | Biol. Chem., |
| | | | | 264: 21522-21528, |
| | | | | 1989 |

TABLE 2
Examples of subcellular localization element sequences.

| Localization | Sequence |
|---|---|
| Nucleus nuclear | C-terminus (28AA) |
| localization | SKKEEKGRSKKEEKGRSKKEEKGRIHRI* |
| signal (NLS) of | (SEQ ID NO: 32) |
| the simian virus | tccaaaaaagaagagaaaggtagatccaaaaaagaagagaaaggtagatccaaaaaagaaga |
| 40 large T- | gaaaggtaggatccaccggatctag |
| antigen | (SEQ ID NO: 33) |
| Golgi the N- | N-terminus (89AA) |
| terminal 81 | MRLREPLLSGSAAMPGASLQRACRLLVAVCALHLGVTLVYY |
| amino acids of | LAGRDLSRLPQLVGVSTPLQGGSNSAAAIGQSSGELRTGGAK |
| human beta 1,4- | DPPVAT |
| galactosyl- | (SEQ ID NO: 34) |
| transferase (GT) | atgaggcttcgggagccgctcctgagcggcagcgccgcgatgccaggcgcgtccctacagcg |
| | ggcctgccgcctgctcgtggccgtctgcgctctgcaccttggcgtcaccctcgtttactacctggc |
| | tggccgcgacctgagccgcctgccccaactggtcggagtctccacaccgctgcagggcggctc |
| | gaacagtgccgccgccatcgggcagtcctccggggagctccggaccggaggggccaaggat |
| | ccaccggtcgccacc |
| | (SE ID NO: 35) |
| Mitochondria | N-terminus (36AA) |
| targeting | MSVLTPLLLRGLTGSARRLPVPRAKIHSLGDPPVAT |
| sequence derived | (SEQ ID NO: 36) |
| from the | atgtccgtcctgacgccgctgctgctgcggggcttgacaggctcggcccggcggctcccagtg |
| precursor of | ccgcgcgccaagatccattcgttgggggatccaccggtcgccacc |
| subunit VIII of | (SEQ ID NO: 37) |
| human | |
| Cytochrome C | |
| oxidase | |
| ER targeting | N-terminus (18AA) |
| sequence of | MLLSVPLLLGLLGLAVAV |
| calreticulin | (SEQ ID NO: 38) |
| | atgctgctatccgtgccgttgctgctcggcctcctcggcctggccgtcgccgtg |
| | (SEQ ID NO: 39) |

### VIII. DNA Constructs, Expression Vectors and Host Cells

Nucleic acid molecules encoding one or more test proteins, SFP detectors, tags, and fusions of two of more thereof can be included in one or more expression vectors to direct expression of the corresponding nucleic acid sequence. Thus, other expression control sequences including appropriate promoters, enhancers, transcription terminators, a start codon at the front of a protein-encoding sequence, splicing signal for introns, maintenance of the correct reading frame of that gene to permit proper translation of mRNA, and stop codons can be included in an expression vector. Generally, expression control sequences include a promoter, a minimal sequence sufficient to direct transcription.

Nucleic acid sequences encoding test proteins, SFP tags, SFP detectors and fusions of two or more thereof, etc., may be included in an expression vector to direct expression of the corresponding nucleic acid sequence. Optionally, the nucleic acid sequences encoding an SFP tag, affinity tag and/or SFP detector may be operably linked to the nucleic acid encoding a test protein, such that expression from the expression vector results in a fusion protein of the test protein fused to the SFP tag, affinity tag and/or SFP detector.

As will be appreciated by the skilled artisan, expression vectors used to express test proteins, SFP tags, affinity tags, SFP detectors and fusions thereof must be compatible with the host cell in which the proteins are to be expressed. Similarly, various promoter systems are available and should be selected for compatibility with cell type, strain, etc. Codon optimization techniques may be employed to adapt sequences for use in other cells, as is well known.

The expression vector typically contains an origin of replication, a promoter, as well as specific genes which allow phenotypic selection of the transformed cells (e.g., an antibiotic resistance cassette). Vectors suitable for use include, but are not limited to, the pMSXND expression vector for expression in mammalian cells (Lee and Nathans, J. Biol. Chem. 263:3521, 1988). Generally, the expression vector will include a promoter. The promoter can be inducible or constitutive. In one embodiment, the promoter is a heterologous promoter.

Unlike constitutive promoters, an inducible promoter is not always active. Some inducible promoters are activated by physical stimuli, such as the heat shock promoter. Others are activated by chemical stimuli, such as IPTG or Tetracycline (Tet), or galactose. Inducible promoters or gene-switches are used to both spatially and temporally regulate gene expression. Thus, for a typical inducible promoter in the absence of the inducer, there would be little or no gene expression while, in the presence of the inducer, expression should be high (i.e., off/on). The skilled artisan is familiar with inducible promoters and will appreciate which inducible promoters may be used in the embodiments described herein.

In some embodiments, multiple inducible promoters are included on an expression vector, each promoter induced by a different inducer. In other embodiments, multiple expression vectors are included in the host cell, each expression vector comprising an inducible promoter, each inducible promoter induced by a different inducer. In this way, expression of multiple proteins in a host cell can be independently under the control of separate inducible promoters. Thus, in some embodiments, host cells are engineered to express one or more complementary fragments of a SFP, one or more of which are fused to one or more test proteins. The fragments may be expressed simultaneously or sequentially.

Systems of two independently controllable promoters have been described and are well known in the art, and are described herein. See, for example, Lutz and Bujard, Nucleic Acids Res., 25:1203-1210, 1997.

In one example, a vector in which the promoter is under the repression of the Laclq protein and the arabinose inducer/repressor may be used for expression of the SFP detector (e.g., pPROLAR vector available from Clontech, Palo Alto, Calif.). Repression is relieved by supplying IPTG and arabinose to the growth media, resulting in the expression of the SFP detector. In this system, the araC repressor is supplied by the genetic background of the host E. coli cell. For the controlled expression of the test protein-SFP tag fusion, a vector in which the test protein-SFP tag fusion is under the repression of the tetracycline repressor protein may be used (e.g., pPROTET vector; Clontech). In this system, repression is relieved by supplying anhydrotetracycline to the growth media, resulting in the expression of the test protein-SFP tag fusion construct. The tetR and Laclq repressor proteins may be supplied on a third vector, or may be incorporated into the fragment-carrying vectors.

In one example, nucleic acid encoding a test protein, SFP tag, SFP detector or fusion of two or more thereof is located downstream of the desired promoter. Optionally, an enhancer element is also included, and can generally be located anywhere on the vector and still have an enhancing effect. However, the amount of increased activity will generally diminish with distance. Expression vectors including a nucleic acid encoding a test protein, SFP tag, SFP detector or fusion of two or more thereof can be used to transform host cells.

The disclosed embodiments may be applied in virtually any host cell type, including without limitation bacterial cells (e.g., E. coli) and mammalian cells (e.g., CHO cells). Hosts can include isolated microbial, yeast, insect and mammalian cells, as well as cells located in the organism. For example, the host cell may be an E. coli cell, such as an E. coli BL21 (DE3) strain cell. Secretion competent yeast and bacterial cells may be used. The skilled artisan is familiar with such cells. Nucleic acid encoding test proteins, affinity tags, SFP tags, SFP detectors and fusion proteins are typically comprised in an expression vector introduced into the host cells. One limitation is that expression of GFP and GFP-like proteins is compromised in highly acidic environments (i.e., pH=4.0 or less). Likewise, complementation rates are generally inefficient under conditions of pH of 6.5 or lower (see, e.g., U.S. patent application Ser. No. 10/973,693).

A transfected cell is a cell into which (or into an ancestor of which) has been introduced, by means of recombinant DNA techniques, a DNA molecule encoding a protein of interest. Transfection of a host cell with recombinant DNA may be carried out by conventional techniques as are well known in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such as a CHO cell, such methods of transfection of DNA as calcium phosphate coprecipitates, conventional mechanical procedures such as microinjection, electroporation, insertion of a plasmid encased in a liposome, or virus vectors may be used. Eukaryotic cells can also be cotransformed with DNA sequences encoding the test protein, and a second foreign DNA molecule encoding a selectable phenotype, such as neomycin resistance. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). Other specific, non-limiting examples of viral vectors include adenoviral vectors, lentiviral vectors, retroviral vectors, and pseudorabies vectors.

As will be appreciated by those skilled in the art, the vectors used to express the test proteins, SFP tags, SFP detectors and fusions of two or more thereof disclosed herein must be compatible with the host cell in which the vectors are provided. Similarly, various promoter systems are available and should be selected for compatibility with cell type, strain, etc. Codon optimization techniques may be employed to adapt sequences for use in other cells, as is well known. In some examples, expression of polypeptides may be performed using a cell-free system; such systems are known to the skilled artisan and are commercially available (see, e.g., Cat No. K9901-01, Invitrogen, Corp., Carlsbad, Calif.).

When using mammalian cells for the subcellular localization methods described herein, an alternative to codon optimization is the use of chemical transfection reagents, such as the recently described chariot system (Morris et al., Nature Biotechnol. 19: 1173-1176, 2001). The Chariot™ protein delivery reagent (Activmotif, Corp., Carlsbad, Calif.) may be used to directly transfect a protein into the cytoplasm of a mammalian cell. Thus, this approach would be useful for providing a SFP fragment (e.g., an SFP detector) within a host cell, for instance before, after or during expression of a complementary SFP fragment expressed within the host cell.

### IX. Kits

Provided herein are kits useful for the various embodiments described herein. The kits may facilitate the use of SFPs for determining the subcellular localization of a protein as described herein. Kits may contain various materials and reagents (e.g., for practicing the methods described herein). For example, a kit may contain reagents including, without limitation, polypeptides or polynucleotides, cell transformation and transfection reagents, reagents and materials for purifying polynucleotides and polypeptides including lysis regents, protein denaturing and refolding reagents, as well as other solutions or buffers useful in carrying out the assays and other methods of the invention. Kits may also include control samples, materials useful in calibrating methods described herein, and containers, tubes, microtiter plates and the like in which assay reactions may be conducted. Kits may be packaged in containers, which may comprise compartments for receiving the contents of the kits, instructions for conducting methods described herein or using the polypeptides and polynucleotides described herein, etc.

For example, a kit may provide one or more SFP fragments as described herein, one or more polynucleotide constructs encoding the one or more SFP fragments, one or more polynucleotide constructs encoding one or more subcellular localization elements as described herein, cell strains suitable for propagating the constructs, cells pre-transformed or stably transfected with constructs encoding one or more SFP fragments, and reagents for purification of expressed fusion proteins or nucleotide encoding an expressed fusion protein. For example, a kit may provide a nucleic acid construct encoding a SFP tag and a multiple cloning site adjacent thereto, such that an encoding sequence inserted into the multiple cloning site results in a nucleic acid that encodes a protein encoded by the encoding sequence fused with the SFP tag, and instructions for using the nucleic acid (e.g., instructions for carrying out the methods described herein). In another example, a kit may provide a nucleic acid construct encoding a SFP detector as described herein and a multiple cloning site adjacent thereto, such that an encoding sequence inserted into the multiple cloning site results in a nucleic acid molecule that encodes a protein encoded by the encoding sequence fused with the SFP detector and instructions for using the nucleic acid (e.g., instructions for carrying out the methods described herein).

In one embodiment of a kit, the kit includes a nucleic acid construct containing the coding sequence of a SFP tag (e.g., GFP S11) and a multiple cloning site for inserting a test protein in-frame at the N- or C-terminus of the SFP tag coding sequence. Optionally, the insertion site may be followed by the coding sequence of a linker polypeptide in frame with the coding sequence of the downstream SFP tag sequence. A specific embodiment is the pTET-SpecR plasmid as described in U.S. Pat. App. Pub. No. 2005/0221343. This nucleic acid construct may be used to produce test protein-SFP tag fusions in suitable host cells.

In some embodiments, a kit includes a nucleic acid construct containing the coding sequence of a SFP detector as described herein (e.g., GFP S1-10, CFP S1-10 or YFP S1-10) and a multiple cloning site for inserting a test protein in-frame at the N- or C-terminus of the SFP tag coding sequence. For example, the kit may include a nucleic acid construct encoding a SFP detector as set forth as any of SEQ ID NO: 18, SEQ ID NO: 19, SEQ ID NO: 20, SEQ ID NO: 21, SEQ ID NO: 22, SEQ ID NO: 23, SEQ ID NO: 25, SEQ ID NO: 26, SEQ ID NO: 27, SEQ ID NO: 28, SEQ ID NO: 29, SEQ ID NO: 30 or SEQ ID NO: 31. In one example, the kit includes one or more nucleic acid constructs encoding a SFP detector as set forth as any of SEQ ID NO: 18, SEQ ID NO: 19, SEQ ID NO: 20, SEQ ID NO: 21, SEQ ID NO: 22, SEQ ID NO: 23, SEQ ID NO: 25, SEQ ID NO: 26, SEQ ID NO: 27, SEQ ID NO: 28, SEQ ID NO: 29, SEQ ID NO: 30 or SEQ ID NO: 31, each in a separate container or vial, wherein the nucleic acid coding sequence may be part of a vector. Optionally, the insertion site may be followed by the coding sequence of a linker polypeptide in frame with the coding sequence of the downstream SFP tag sequence. A specific embodiment is the pTET-SpecR plasmid as described in U.S. Pat. App. Pub. No. 2005/0221343.

In some embodiments, the kit further contains a pre-purified SFP detector (e.g., GFP S1-10, YFP S1-10 or CFP S1-10 polypeptide) used to detect test protein-SFP tag fusions. In some examples, the purified SFP detector is fused to a subcellular localization element.

### EXAMPLES

The following examples are provided to illustrate certain particular features and/or embodiments and should not be construed as limiting.

### Example 1

### Addition of the Y66W Substitution to Split-GFP Results in a Non-Functional Split-CFP

This example describes incorporation of the Y66W substitution into the Split-GFP S1-10 SFP detector. The Y66W substitution was originally identified as a substitution that, when incorporated into GFP, results in a fluorescent molecule with blue-shifted excitation and emission characteristics, commonly known as CFP. Thus, it was expected that incorporation of the Y66W substitution into the Split-GFP S1-10 detector would result in a SFP detector that, when complemented with a SFP tag, would result in a Split fluorescent molecule corresponding to CFP. However, as described below, incorporation of the Y66W substation into the Split-GFP S1-10 detector results in a SFP detector that, when complemented with a SFP tag, results in a non-functional fluorescent molecule.

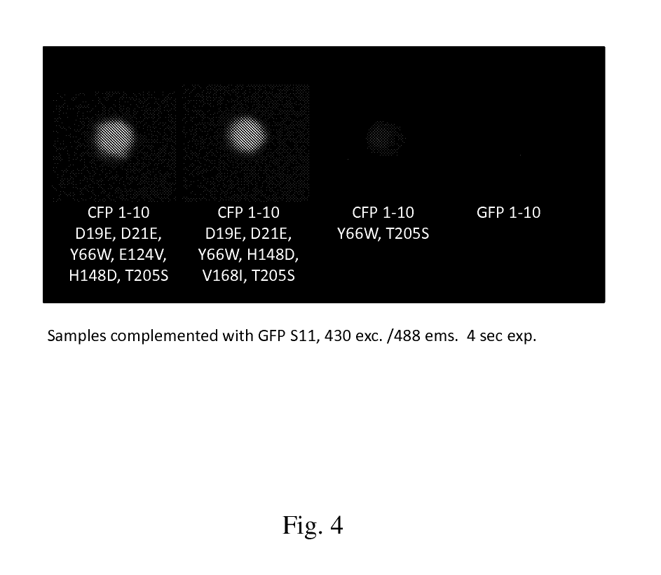

Construction of GFP S-10 with the Y66W substitution was done with a conventional PCR assembly reaction. Incorporation of the Y66W substitution into the amino acid sequence of Split-GFP S1-10 (SEQ ID NO: 4) results in a polypeptide fragment that, when complemented with a GFP S11 tag (SEQ ID NO: 16), does not produce a significant cyan fluorescent signal. As shown in FIG. 4, after complementation with the GFP S11 tag (SEQ ID NO: 16) a polypeptide with the Y66W substitution into the amino acid sequence of Split-GFP S1-10 (SEQ ID NO: 4) did not produce a significant fluorescent signal at 488 nm when excited with 430 nm light. Thus, unexpectedly, incorporation of the conventional amino acid substitution (Y66W) used to generate CFP into Split-GFP did not result in a functional Split-CFP molecule.

### Example 2

### Engineering Functional Split-CFPs

This example describes the development of functional Split-CFP molecules. A directed evolution screen was conducted to identify possible Split-CFPs, using GFP S1-10 Y66W as the starting point for the screen. The results of the screen identified novel polypeptide which complement with GFP S11 (SEQ ID NO: 16) to form a functional Split-CFP.

### Directed Evolution of GFP S1-10 to Develop Split-CFP Fragments

A directed evolution strategy was used to develop Split-CFP fragments using Split-GFP 51-10 (SEQ ID NO: 4) comprising a Y66W substitution as a starting point. The cDNA encoding the Split-GFP 51-10 (SEQ ID NO: 4) comprising a Y66W substitution was subjected to DNA shuffling techniques to generate a library of substitutions (for example, as described in U.S. Pat. App. Pub. No. 2009/0142820). The directed evolution of GFP 51-10 resulted in a series of polypeptides having the protein sequence of GFP S1-10 (SE ID NO: 4), but with the amino acid substitutions listed in Table 3. Additionally, each of polypeptides carries a T216S substitution compared to SE ID NO: 4; this is due to the cloning of a nucleotide sequence encoding the GFP S1-10 fragment and the residue at positions 215 and 216 of GFP S1-10 is not needed for a functional SFP detector or to form complementation with a complementary SFP tag.

TABLE 3
Split-CFP substitutions.

| Ident. | Substitutions | | | | | | | | | |
|---|---|---|---|---|---|---|---|---|---|---|
| A1 | | D19E | D21E | Y66W | | E124V | H148D | | T205S | |
| B1 | | D19E | D21E | Y66W | | | H148D | | T205S | |
| C1 | | D19E | D21E | Y66W | | | H148D | V167I | T205S | |
| D1 | | | | Y66W | | | H148D | | T205S | |
| E1 | | D19E | D21E | Y66W | | | H148D | | T205S | |
| F1 | | D19E | D21E | Y66W | | | H148D | | T205S | |
| G1 | | D19E | D21E | Y66W | | | H148D | | T205S | |
| H1 | | D19E | D21E | Y66W | | | H148D | | T205S | |
| A2 | | D19E | D21E | Y66W | | | H148D | | T205S | |
| B2 | | D19E | D21E | Y66W | | | H148D | | T205S | |
| C2 | | D19E | D21E | Y66W | | | H148D | | T205S | |
| D2 | V16I | D19E | D21E | Y66W | | | H148D | | T205S | |
| E2 | | D19E | D21E | Y66W | | | H148D | | T205S | |
| F2 | | D19E | D21E | Y66W | | | H148D | | T205S | |
| G2 | | D19E | D21N | Y66W | | | H148D | | T205S | |
| H2 | | | | Y66W | | | H148D | | T205S | |
| A3 | | D19E | D21E | Y66W | | | H148D | | T205S | |
| B3 | | D19E | D21E | Y66W | | | H148D | | T205S | |
| C3 | | D19E | D21E | Y66W | | | H148D | | T205S | |
| D3 | None | | | | | | | | | |
| E3 | | D19E | D21E | Y66W | | | H148D | | T205S | |
| F3 | | D19E | D21E | Y66W | | | H148D | | T205S | |
| G3 | | D19E | D21E | Y66W | | | H148D | | T205S | |
| H3 | | D19E | D21N | Y66W | | | H148D | | T205S | |
| B4 | | D19E | D21E | Y66W | | | H148D | | T205S | |
| C4 | | D19E | D21E | Y66W | | | H148D | | T205S | S208L |
| D4 | | | | Y66W | | | H148D | | T205S | |
| E4 | | | | Y66W | | | H148D | | T205S | |
| F4 | | D19E | D21E | Y66W | | | H148D | | T205S | |
| G4 | | | D21E | Y66W | | | H148D | | T205S | |
| H4 | | D19E | D21E | Y66W | | | H148D | | T205S | |
| A5 | | D19E | | Y66W | | | H148D | | T205S | |
| B5 | | D19E | D21E | Y66W | | | H148D | | T205S | |
| C5 | | D19E | D21E | Y66W | | | H148D | | T205S | |
| D5 | | D19E | D21E | Y66W | | | H148D | | T205S | |
| E5 | | D19E | D21E | Y66W | | | H148D | | T205S | |
| F5 | | D19E | D21E | Y66W | | | H148D | | T205S | |
| G5 | | D19E | D21E | Y66W | | | H148D | | T205S | S208L |
| H5 | | D19E | D21N | Y66W | | | H148D | | T205S | |
| A6 | | D19E | D21E | Y66W | | | H148D | | T205S | |
| B6 | | D19E | D21E | Y66W | | | H148D | | T205S | |
| C6 | | D19E | D21N | Y66W | | | H148D | | T205S | |
| D6 | | | | Y66W | | | H148D | | T205S | |
| E6 | | D19E | D21E | Y66W | | | H148D | | T205S | |
| F6 | | D19E | D21E | | | | H148D | | T205S | |
| G6 | | D19E | D21E | Y66W | | | H148D | | T205S | |
| H6 | | | D21E | | | | X | | T205S | |
| A7 | | D19E | D21N | Y66W | | | H148D | | T205S | |
| C7 | | D19E | D21N | Y66W | | | H148D | | T205S | |
| D7 | | D19E | D21N | Y66W | | | H148D | | T205S | |
| E7 | | D19E | D21E | Y66W | | | X | | T205S | |
| F7 | | D19E | D21E | Y66W | | | H148D | | T205S | |
| G7 | | D19E | D21E | Y66W | | | H148D | | T205S | |
| H7 | | D19E | D21E | Y66W | | | H148D | | T205S | |
| A8 | | D19E | D21E | Y66W | | | H148D | | T205S | |
| B8 | V16I | D19E | D21E | Y66W | | | H148D | | T205S | |
| C8 | | D19E | D21N | Y66W | | | H148D | | T205S | |
| D8 | | | | Y66W | | | H148D | | T205S | |
| E8 | | D19E | D21N | Y66W | | | H148D | | T205S | |
| F8 | | | | Y66W | | | H148D | | T205S | |
| G8 | | D19E | D21N | Y66W | | | H148D | | T205S | |
| H8 | | D19E | D21E | Y66W | | | H148D | | T205S | |
| A9 | | D19E | D21E | Y66W | | | H148D | | T205S | |
| B9 | | D19E | D21N | Y66W | S99T | | H148D | | T205S | |
| C9 | | D19E | D21E | Y66W | | | H148D | | T205S | |
| D9 | | D19E | D21E | Y66W | | | H148D | | T205S | |
| F9 | | D19E | D21E | Y66W | | | H148D | | T205S | |
| G9 | | D19E | D21N | Y66W | | | H148D | | T205S | |
| H9 | | D19E | D21N | Y66W | | | H148D | | T205S | |
| A10 | | D19E | D21E | Y66W | | | H148D | | T205S | |
| B10 | V16I | D19E | D21E | Y66W | | | H148D | | T205S | |
| C10 | | D19E | D21N | Y66W | | | H148D | | T205S | |
| D10 | | D19E | D21N | Y66W | | | H148D | | T205S | |
| A12 | | | D21N | Y66W | | | H148D | | T205S | |
| B12 | | | D21N | Y66W | | | H148D | | T205S | |
| C12 | | D19E | D21N | Y66W | S99T | | H148D | | T205S | |
| D12 | | D19E | D21E | Y66W | | | H148D | | T205S | |
| E12 | V16I | | D21E | Y66W | | | | | T205S | |
| F12 | | | D21N | Y66W | | | | | T205S | |
| G12 | | | | Y66W | | | | | T205S | |
| H12 | | | | Y66W | | | | | T205S | |

### Functional Assays to Identify Split-CFP Molecules

The complementation of the molecules listed in Table 3 with a GFP S11 tag (SEQ ID NO: 16) was examined using a kinetic assay to identify clones that will complement with a SFP tag to form a functional Split-CFP molecule.

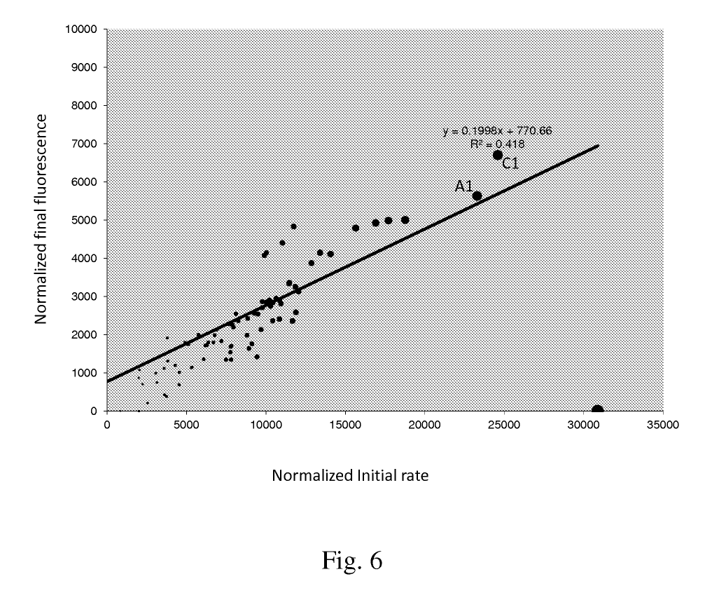

Kinetic assays were performed as described in Listwan et al. (J. Struct. Funct. Genomics, 10:47-55, 2009). Briefly, the CFP1-10 mutants and controls were cloned into a pTET vector encoding N-terminal 6H is tag under control of an AnTET-controlled promoter, such that AnTET-induced expression from the vector results in a 6His-CFP S1-10 fusion protein, and transformed into chemically competent E. coli. A 96-well plate of the E. ColiCFP 1-10 was grown out, induced with AnTET, arrested with 1 mM Chlorimphenicol, and lysed via sonication. 40 ul of the supernatant (soluble fraction) was assayed with a vast excess of purified sulfite reductase tagged with GFP S11 (SEQ ID NO: 16). The fluorescence at 400 nm wavelength excitation and 530 nm wavelength emission was measured every 90 seconds for approximately 8 hours using a fluorescent plate reader. This data was used to calculate the initial rate of fluorescence (see Table 2). Additionally, a final fluorescent reading was taken at 16 hours after complementation (see Table 2). Values were normalized according to sample absorbance and the normalized initial rate and final fluorescence were then graphed on an XY scatter plot (see FIG. 6). As shown in FIG. 6, the two CFP 1-10 optima (1 and 3 in spreadsheet, corresponding to A1 and C1 respectively; discussed below) presented as the two clones having the highest initial rate and final fluorescence.

TABLE 4
Final fluorescence and initial rate measurements for Split-CFP
substitutions.

| | Final | Initial |
|---|---|---|
| Ident. | Fluorescence | Rate |
| A1 | 5633.537 | 23287.1 |
| B1 | 4790.074 | 15651.14 |
| C1 | 6700.685 | 24586.52 |
| D1 | 8864.456 | 14476.46 |
| E1 | 4830.722 | 11742.92 |
| F1 | 4070.275 | 9888.74 |
| G1 | 4403.01 | 11034.48 |
| H1 | 4141.501 | 10022.99 |
| A2 | 4110.639 | 14069.03 |
| B2 | 3255.459 | 11850.7 |
| C2 | 2253.8 | 7886.102 |
| D2 | 3362.49 | 11474.6 |
| E2 | 1688.021 | 7778.341 |
| F2 | 1795.408 | 6694.27 |
| G2 | 1913.043 | 3787.626 |
| H2 | 6567.077 | 9057.327 |
| A3 | 1988.754 | 8808.068 |
| B3 | 2359.545 | 8259.187 |
| C3 | 2197.111 | 7952.574 |
| D3 | 2518.183 | 2582.434 |
| E3 | 1983.486 | 6765.691 |
| F3 | 2286.555 | 7599.914 |
| G3 | 2407.039 | 10842.52 |
| H3 | 2134.243 | 6873.214 |
| A4 | 4146.745 | 13408.6 |
| B4 | 1760.689 | 9111.157 |
| C4 | 2271.854 | 7793.284 |
| D4 | 3735.943 | 7994.234 |
| E4 | 3276.064 | 7276.295 |
| F4 | 1542.654 | 7758.682 |
| G4 | 5103.892 | 13014.25 |
| H4 | 2362.952 | 10420.62 |
| A5 | 2863.587 | 9766.459 |
| B5 | 2742.581 | 10285.25 |
| C5 | 2538.627 | 9488.02 |
| D5 | 2424.075 | 8848.268 |
| E5 | 1795.348 | 6365.505 |
| F5 | 2900.564 | 10210.69 |
| G5 | 1358.783 | 6074.743 |
| H5 | 1995.025 | 5749.581 |
| A6 | 5002.239 | 18759.45 |
| B6 | 2946.64 | 10636.21 |
| C6 | 1117.322 | 3586.843 |
| D6 | 4097.623 | 10046.55 |
| E6 | 2560.074 | 9260.172 |
| F6 | 2909.285 | 10854.8 |
| G6 | 2584.609 | 11882.27 |
| H6 | 3374.937 | 7123.464 |
| A7 | 2545.536 | 8103.773 |
| B7 | 234.8107 | 0 |
| C7 | 1728.355 | 6279.593 |
| D7 | 744.0754 | 3123.287 |
| E7 | 2840.368 | 10428.21 |
| F7 | 2849.845 | 10008.89 |
| G7 | 2133.18 | 9682.955 |
| H7 | 3874.809 | 12859.26 |
| A8 | 2359.895 | 11667.06 |
| B8 | 1638.985 | 8925.641 |
| C8 | 1013.726 | 4545.283 |
| D8 | 3753.761 | 8436.234 |
| E8 | 1140.234 | 5325.679 |
| F8 | 1313.746 | 3818.533 |
| G8 | 1418.792 | 9435.207 |
| H8 | 2815.422 | 10931.37 |
| A9 | 4985.972 | 17710.66 |
| B9 | 702.9612 | 2230.034 |
| C9 | 1834.316 | 7195.78 |
| D9 | 1344.75 | 7486.497 |
| E9 | 690.0848 | 4530.79 |
| F9 | 2708.828 | 9774.605 |
| G9 | 1703.044 | 7817.784 |
| H9 | 1344.235 | 7798.565 |
| A10 | 4926.375 | 16909.24 |
| B10 | 3339.063 | 11451.53 |
| C10 | 1716.594 | 6178.272 |
| D10 | 1196.82 | 4289.569 |
| E10 | 210.6187 | 2552.979 |
| F10 | 0 | 0 |
| G10 | 0 | 0 |
| H10 | 0 | 0 |
| A11 | 0 | 830.7597 |
| B11 | 0 | 0 |
| C11 | 0 | 1997.507 |
| D11 | 4868.154 | 0 |
| E11 | 1002.297 | 0 |
| F11 | 271.5854 | 0 |
| G11 | 0 | 30877.35 |
| H11 | 219.5189 | 0 |
| A12 | 989.8494 | 3056.43 |
| B12 | 1792.882 | 4901.018 |
| C12 | 1744.922 | 5120.837 |
| D12 | 3129.463 | 12041.21 |
| E12 | 1069.797 | 2038.043 |
| F12 | 867.9638 | 1982.741 |
| G12 | 381.8273 | 3743.39 |
| H12 | 424.8945 | 3602.294 |

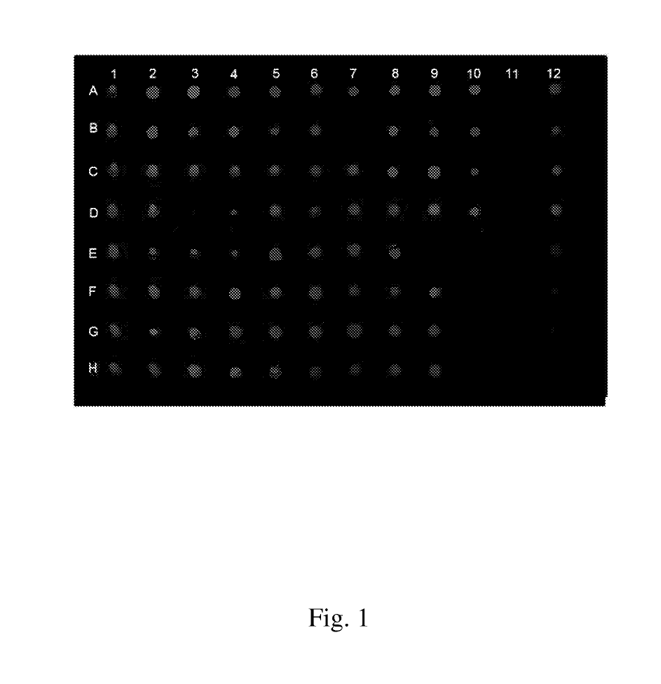

Additionally, E. coli comprising nucleic acid constructs encoding each of the clones listed in Table 3 as well as GFP S11 (SEQ ID NO: 16) were grown and expression of the clone listed in table 3 and GFP S11 induced. Briefly, the CFP1-10 mutants and controls were cloned into a pTET vector encoding N-terminal 6H is tag under control of an AnTET-controlled promoter, such that AnTET-induced expression from the vector results in a 6His-CFP S1-10 fusion protein, and transformed into chemically competent E. coli. The E. coli were previously transformed with a second pET vector coding for sulfite reductase tagged with a C-terminal GFP S11 (SEQ ID NO: 16) under the control of a IPTG-inducible promoter. Using a 96 well replication tube, clones were plated on nitrocellulose membranes resting on LB agar (growth media) and grown for 16 hours at 30° C. Protein expression from the CFP S1-10 pTET vector was induced by moving the nitrocellulose membrane to media containing 3 μg/ml of anhydrous tetracycline (AnTET) for 1.5 hours at 37° C. Cells were returned to the growing media and incubated at 37° C. for 1 hour to allow the AnTET to diffuse out of the cells. Protein expression from the second pET vector coding for sulfite reductase tagged with a C-terminal GFP S11 (SEQ ID NO: 16) was then induced by moving the nitrocellulose membrane to media containing 1 mM IPTG for one hour at 37° C. Any resulting functional Split-CFP molecule was identified by detecting fluorescence emitted from the bacteria at 488 nm wavelength when excited with 430 nm wavelength light. The sequential expression protocol used herein prevents false-positive solubility results because CFP S1-10 clones must remain are unable to complement with the S11 fragment until that fragment is expressed. As shown in FIG. 1, several individual members of the set of Split-CFP mutants developed using the directed evolution strategy described above exhibited Split-CFP fluorescent properties.

### Split-CFP Optima

The directed evolution screen and the kinetic assays resulted in the identification of several polypeptides that will complement with GFP S11 (SEQ ID NO: 16) to form a functional Split-CFP molecule. For example, of the polypeptides identified, a GFP S1-10 with D19E, D21E, Y66W, E124V, H148D, T2055 substitutions (SEQ ID NO: 20) or with D19E, D21E, Y66W, H148D, V1671, T2055 substitutions (SEQ ID NO: 21) generated the greatest initial rate and final fluorescence at the 488 nm wavelength channel when complemented with a SFP tag and excited at 430 nm light. An example of the fluorescence of these molecules is shown in FIG. 4.

### Example 3

### Addition of the T203Y Substitution to Split-GFP Results in a Non-Functional Split-YFP

This example describes incorporation of the T203Y substitution into the Split-GFP S1-10 SFP detector. The T203Y substitution was originally identified as a substitution that, when incorporated into GFP, results in a fluorescent molecule with red-shifted excitation and emission characteristics, commonly known as YFP, which has excitation and emission characteristics distinct from GFP. Thus, it was expected that incorporation of the T203Y substitution into the Split-GFP S1-10 detector would result in a SFP detector that, when complemented with a SFP tag, would result in a SFP corresponding to YFP. However, as described below, incorporation of the T203Y substation into the Split-GFP S1-10 detector results in a SFP detector that, when complemented with a SFP tag, lacks excitation and emission characteristics significantly distinct from Split-GFP.

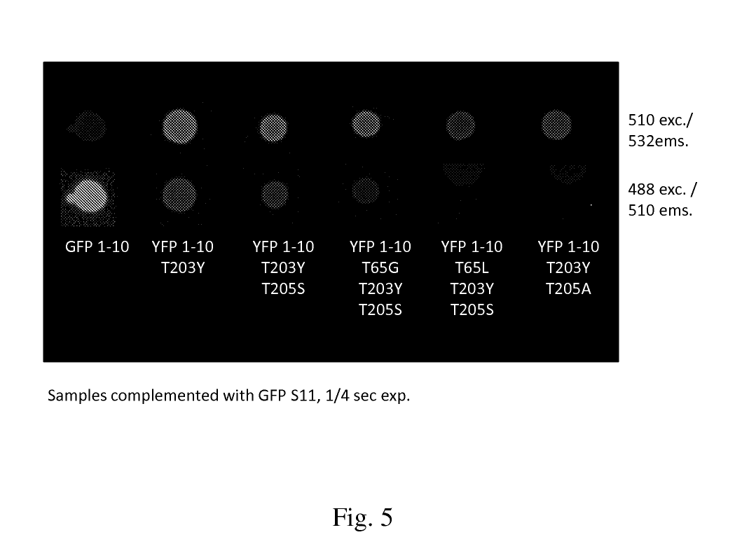

Construction of GFP S-10 with the T203Y substitution was done with a conventional PCR assembly reaction. Incorporation of the T203Y substitution into the amino acid sequence of Split-GFP S1-10 (SE ID NO: 4) results in a polypeptide fragment that, when complemented with a GFP S11 tag (SEQ ID NO: 16), does not produce a yellow fluorescent signal that is significantly differentiated from the Split-GFP signal. As shown in FIG. 5, after complementation with the GFP S11 tag (SEQ ID NO: 16) a polypeptide with the Y66W substitution into the amino acid sequence of Split-GFP S1-10 (SE ID NO: 4) did not produce a significantly different fluorescent signal at 510 nm or 532 nm compared to Split-GFP when excited with 488 or 510 nm light respectively. Thus, unexpectedly, incorporation of the conventional amino acid substitution (T203Y) used to generate YFP into Split-GFP did not result in a functional Split-YFP molecule.

### Example 4

### Engineering Functional Split-YFPs

This example describes the development of functional Split-YFPs. A degenerate library screen of substitutions at specific residues of GFP S1-10 (SE ID NO: 4) was conducted, using GFP S1-10 T203Y as the starting point for the screen. The results of the screen identified novel polypeptide which complement with GFP S11 (SEQ ID NO: 16) to form a functional Split-YFP.

### Degenerate Libraries of GFP S1-10 to Develop Split-YFP Fragments

A degenerate library of Split-YFP S1-10 substitutions was constructed using Split-GFP S1-10 (SE ID NO: 4) comprising a T203Y substitution as a starting point. First, PCR assembly with variant primers was used to generate diversity at amino acid residues 65 and 205 of Split-GFP 51-10 (SE ID NO: 4). Second, a directed evolution strategy was performed according to known methods (for example, as described in U.S. Pat. App. Pub. No. 2009/0142820) to increase the diversity of the library. The degenerate library screen resulted in a series of polypeptides having the protein sequence of GFP S1-10 (SE ID NO: 4), but with the amino acid substitutions listed in Table 3. Additionally, each of polypeptides carries a T216S substitution compared to

SE ID NO: 4; this is due to the cloning of a nucleotide sequence encoding the GFP S1-10 fragment and the residue at positions 215 and 216 of GFP S1-10 is not needed for a functional SFP detector or to form complementation with a complementary SFP tag.

TABLE 5
Split-YFP substitutions.

| Ident. | Substitutions | | | | | | | | | | | |
|---|---|---|---|---|---|---|---|---|---|---|---|---|
| A1 | | T65L | | | | | | | T203Y | | T205S | |
| B1 | | | | | | | | | | T203Y | | T205S |
| C1 | N/A | | | | | | | | | | | |
| D1 | | | | | | | | | T203Y | | | |
| E1 | | | | | | | | | T203Y | | | |
| F1 | | | | | | | | | T203Y | | | |
| G1 | N/A | | | | | | | | | | | |
| H1 | | | | R80K | | | | | T203Y | | | |
| A2 | | T65L | | | | | | | T203Y | | T205S | |
| B2 | | T65G | | | | | | | T203Y | | | |
| C2 | | T65L | | | | | | | T203Y | | T205S | |
| D2 | | | | | | | | | T203Y | | | |
| E2 | | T65G | | | | | | | T203Y | | T205S | |
| F2 | | T65G | | | | | | | T203Y | | T205S | |
| G2 | | | | | | | | | T203Y | | T205S | |
| H2 | N/A | | | | | | | | | | | |
| A3 | | | | | | | | | T203Y | | T205A | |
| B3 | | | | | | | | | T203Y | | | |
| C3 | | T65G | | | | | | | T203Y | | T205S | |
| D3 | | T65G | | | | | P192H | | T203Y | | T205S | |
| E3 | | | | | | | | | T203Y | | | |
| F3 | | | C70S | | | | | | T203Y | | | |
| G3 | N/A | | | | | | | | | | | |
| H3 | N/A | | | | | | | | | | | |
| A4 | N/A | | | | | | | | | | | |
| B4 | | | | | | | | | T203Y | | | |
| C4 | | T65A | | | | | | | T203Y | | T205S | |
| D4 | | | | | | | | | T203Y | | T205S | |
| E4 | | | | | | | | | T203Y | | | |
| F4 | | T65G | | | | | | | T203Y | | T205S | |
| G4 | N/A | | | | | | | | | | | |
| H4 | | | | | | | | | T203Y | | | |
| A5 | N/A | | | | | | | | | | | |
| B5 | | T65G | | | | | | | T203Y | | T205S | |
| C5 | T9N | T65L | | | | | | | T203Y | | T205S | |
| D5 | | | | | | | | | T203Y | | | |
| E5 | | | | | | | | | T203Y | | T205S | |
| F5 | | T65L | | | | | | | T203Y | | T205S | |
| G5 | | T65L | | | | | | | T203Y | | T205S | |
| H5 | | | | | | | | | T203Y | | | |
| A6 | | | | | | | | | T203Y | | | |
| B6 | | | | | | | | | T203Y | | | |
| C6 | | | | | | | | | T203Y | Q204E | | |
| D6 | | | | | | | | | T203Y | | T205S | |
| E6 | | | | | | | | | T203Y | | | |
| F6 | | | | | | | | | T203Y | | | |
| G6 | | T65G | | | | | | | T203Y | | T205S | |
| H6 | | T65G | | | | | | | T203Y | | T205S | |
| A7 | | T65L | | | | V176I | | | T203Y | | T205S | |
| B7 | N/A | | | | | | | | | | | |
| C7 | | T65G | | | | | | | T203Y | | T205S | |
| D7 | | | | | | | | | T203Y | | | |
| E7 | | T65G | | | | | | | T203Y | | | |
| F7 | | | | | | | | | T203Y | | | |
| G7 | N/A | | | | | | | | | | | |
| H7 | N/A | | | | | | | | | | | |
| A8 | | | | | | | | | T203Y | | | |
| B8 | N/A | | | | | | | | | | | |
| C8 | | | | | | | | | T203Y | | T205S | |
| D8 | | T65G | | | | | | | T203Y | | T205S | |
| E8 | | | | | | | | | T203Y | | | |
| F8 | | | | | | | | | T203Y | Q204H | | |
| G8 | | T65G | | | | | | | T203Y | | T205S | |
| H8 | N/A | | | | | | | | | | | |
| A9 | | T65G | | | | | | | T203Y | | | |
| B9 | | | | | | | | Y200F | T203Y | | | |
| C9 | | | | | | | | | T203Y | | | |
| D9 | | | | | | | | | T203Y | | | |
| E9 | | | | | | | | | T203Y | | | |
| F9 | | | | | | | | | T203Y | | | |
| G9 | | | | | | | | | T203Y | | | |
| H9 | | | | | | | | | T203Y | | | |
| A10 | N/A | | | | | | | | | | | |
| B10 | | | | | | | | | T203Y | | | |
| C10 | | T65L | | | | | | | T203Y | | T205S | |
| D10 | | | | | | | | | T203Y | | | |
| E10 | | | | | | | | | T203Y | | D210V | |
| F10 | | | | | | | | | T203Y | | | |
| G10 | | | | | | | | | T203Y | | | |
| H10 | | | | | | | | | T203Y | | | |
| A11 | | T65G | | | | | | | T203Y | | T205S | |
| B11 | N/A | | | | | | | | | | | |
| C11 | | T65G | | | | | | | T203Y | | T205S | |
| D11 | | | | | | | | | T203Y | | | |
| E11 | | T65G | | | S99F | | | | T203Y | | T205S | |
| F11 | | | | | | | | | T203Y | | | |
| G11 | | | | | S99F | | | | T203Y | | T205S | |
| H11 | | T65L | | | | | | | T203Y | | T205S | |
| H11 | N/A | | | | | | | | | | | |
| A12 | N/A | | | | | | | | | | | |
| B12 | | | | | | | | | T203Y | | T205S | |
| D12 | N/A | | | | | | | | | | | |
| E12 | Cont. | | | | | | | | | | | |
| F12 | Cont. | | | | | | | | | | | |
| G12 | Cont. | | | | | | | | | | | |
| H12 | N/A | | | | | | | | | | | |

### Functional Assays to Identify Split-YFP Molecules.

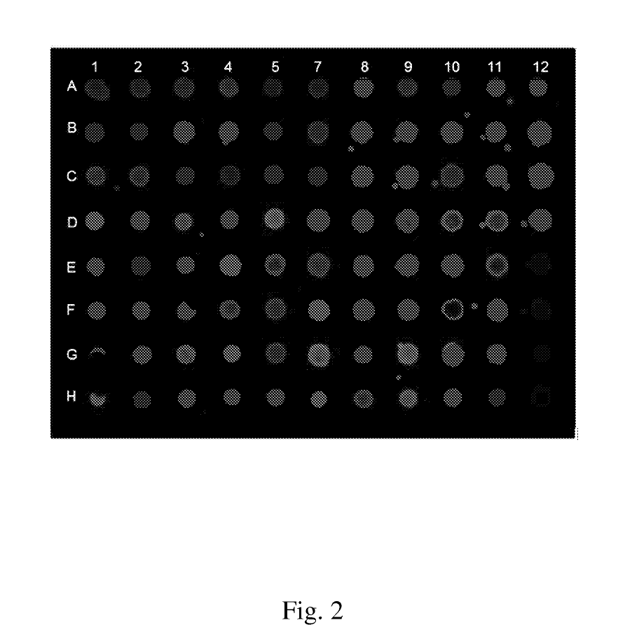

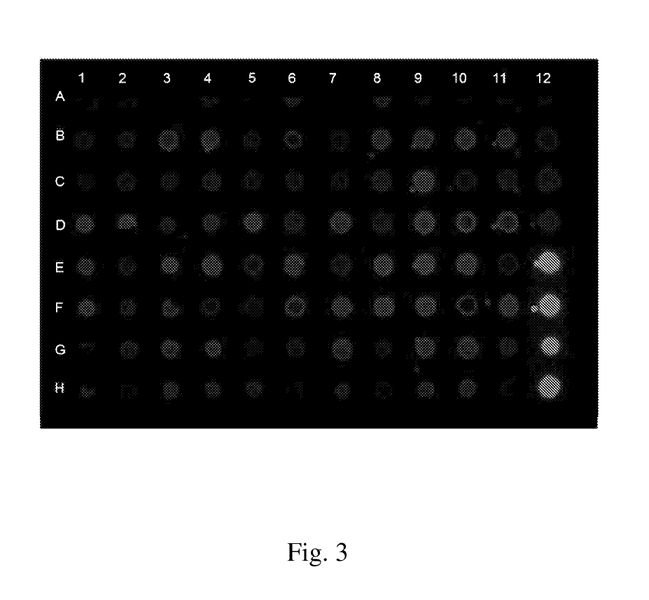

The green and yellow fluorescence of each of the degenerate library clones was tested. E. coli comprising nucleic acid constructs encoding each of the clones listed in Table 3 as well as GFP S11 (SEQ ID NO: 16) were grown and expression of the clones listed in Table 5 and GFP S11 induced. Briefly, the YFP S1-10 mutants and controls were cloned into a pTET vector encoding N-terminal 6H is tag under control of an AnTET-controlled promoter, such that AnTET-induced expression from the vector results in a 6His-YFP S1-10 fusion protein, and transformed into chemically competent E. coli. The E. coli were previously transformed with a second pET vector coding for sulfite reductase tagged with a C-terminal GFP S11 (SEQ ID NO: 16) under the control of a IPTG-inducible promoter. Using a 96 well replication tube, clones were plated on nitrocellulose membranes resting on LB agar (growth media) and grown for 16 hours at 30° C. Protein expression from the YFP S1-10 pTET vector was induced by moving the nitrocellulose membrane to media containing 3 μg/ml of anhydrous tetracycline (AnTET) for 1.5 hours at 37° C. Cells were returned to the growing media and incubated at 37° C. for 1 hour to allow the AnTET to diffuse out of the cells. Protein expression from the second pET vector coding for sulfite reductase tagged with a C-terminal GFP S11 (SEQ ID NO: 16) was then induced by moving the nitrocellulose membrane to media containing 1 mM IPTG for one hour at 37° C. The sequential expression protocol used herein prevents false-positive solubility results because YFP S1-10 clones must remain are unable to complement with the S11 fragment until that fragment is expressed. Any resulting functional Split-YFP molecule was identified by measuring the yellow (Table 6 and FIG. 2) and green (Table 6 and FIG. 3) fluorescent properties of the resulting complemented SFP fragments. The measurement parameters for yellow fluorescence were excitation/emission wavelengths of 510 and 532 nm, respectively. the measurement parameters for green fluorescence were excitation/emission wavelengths of 488 and 510 nm respectively. The ratio of yellow to green fluorescence of the clones was calculated (Table 4). As shown in FIG. 1, several individual members of the set of Split-CFP mutants developed using the directed evolution strategy described above exhibited Split-CFP fluorescent properties.

As shown in Table 6, several clones were identified that emit at least ten-fold greater fluorescence at 532 nm wavelength when excited at 510 nm wavelength than the fluorescence they emit at 510 nm wavelength when excited at 488 nm wavelength under the same conditions.

TABLE 6
Results of fluorescence assays for functional Split-YFP molecules sorted
by ratio of yellow to green fluorescence.

| | Green | Yellow | Ratio |
|---|---|---|---|
| Clone | Fluorescence | Fluorescence | Yellow/Green |
| Identifier | (488/510) | (510/532) | Fluorescence |
| A3 | 1.80488 | 75.7112 | 41.95 |
| H6 | 3.081198 | 124.718 | 40.48 |
| A11 | 3.477405 | 114.015 | 32.79 |
| H8 | 3.853396 | 126.2547 | 32.76 |
| H2 | 3.024467 | 94.4866 | 31.24 |
| A10 | 3.778954 | 117.1476 | 31.00 |
| H11 | 3.43217 | 102.86 | 29.97 |
| A7 | 2.772825 | 79.0921 | 28.52 |
| H11 | 4.249249 | 120.7639 | 28.42 |
| A1 | 3.196619 | 83.46323 | 26.11 |
| G5 | 4.116477 | 105.9474 | 25.74 |
| B5 | 4.7135 | 120.4502 | 25.55 |
| G6 | 5.418159 | 136.716 | 25.23 |
| C7 | 4.813689 | 120.7722 | 25.09 |
| A2 | 3.433795 | 84.90838 | 24.73 |
| A9 | 4.50048 | 109.4179 | 24.31 |
| G8 | 5.611806 | 133.9897 | 23.88 |
| C1 | 4.247373 | 100.0178 | 23.55 |
| G1 | 3.928193 | 91.06359 | 23.18 |
| A4 | 5.101003 | 116.8317 | 22.90 |
| F5 | 4.733509 | 108.2005 | 22.86 |
| A12 | 5.594504 | 127.4514 | 22.78 |
| E11 | 4.510606 | 101.3871 | 22.48 |
| F2 | 5.853969 | 131.1043 | 22.40 |
| B7 | 4.72905 | 105.4578 | 22.30 |
| E2 | 4.712001 | 103.9788 | 22.07 |
| F4 | 5.506557 | 119.8639 | 21.77 |
| A5 | 2.795787 | 60.76794 | 21.74 |
| C3 | 5.621522 | 118.187 | 21.02 |
| B1 | 5.410913 | 113.4642 | 20.97 |
| C5 | 5.071484 | 105.8073 | 20.86 |
| C10 | 5.429226 | 110.4149 | 20.34 |
| C6 | 5.096932 | 101.9792 | 20.01 |
| C2 | 5.342936 | 104.998 | 19.65 |
| B2 | 5.852047 | 114.2019 | 19.51 |
| B12 | 6.637159 | 127.778 | 19.25 |
| C11 | 6.806197 | 129.4402 | 19.02 |
| A6 | 7.738469 | 146.363 | 18.91 |
| G11 | 7.468622 | 140.9211 | 18.87 |
| H1 | 7.412119 | 138.3414 | 18.66 |
| G2 | 7.188156 | 133.3592 | 18.55 |
| D8 | 7.064882 | 130.2324 | 18.43 |
| H5 | 8.767209 | 160.9734 | 18.36 |
| D6 | 6.91 | 124.83 | 18.07 |
| D3 | 7.148303 | 128.249 | 17.94 |
| A8 | 8.536462 | 149.6462 | 17.53 |
| E7 | 6.907653 | 117.2582 | 16.98 |
| H4 | 9.392785 | 155.9727 | 16.61 |
| C8 | 8.118352 | 132.0558 | 16.27 |
| E5 | 7.202748 | 112.5315 | 15.62 |
| H7 | 10.38133 | 161.2327 | 15.53 |
| D12 | 8.68 | 132.21 | 15.23 |
| H3 | 10.83534 | 163.3 | 15.07 |
| F10 | 8.108899 | 115.9812 | 14.30 |
| B6 | 8.08095 | 111.0578 | 13.74 |
| H9 | 12.42045 | 168.1686 | 13.54 |
| H10 | 13.12026 | 174.5189 | 13.30 |
| D4 | 10.61586 | 138.1816 | 13.02 |
| F6 | 11.48465 | 144.1293 | 12.55 |
| G3 | 12.84726 | 154.0704 | 11.99 |
| B11 | 13.06343 | 155.1829 | 11.88 |
| F3 | 10.52154 | 120.3569 | 11.44 |
| E1 | 12.29825 | 139.1341 | 11.31 |
| B8 | 14.17327 | 159.6913 | 11.27 |
| G7 | 14.71777 | 164.3726 | 11.17 |
| G4 | 14.90511 | 162.9495 | 10.93 |
| B9 | 14.70029 | 160.299 | 10.90 |
| C4 | 7.166579 | 76.21599 | 10.63 |
| B3 | 13.90101 | 145.7391 | 10.48 |
| B10 | 16.13969 | 164.511 | 10.19 |
| F1 | 15.82025 | 159.3837 | 10.07 |
| E6 | 15.36882 | 154.7283 | 10.07 |
| G10 | 16.96173 | 168.1668 | 9.91 |
| B4 | 15.95714 | 154.8895 | 9.71 |
| D1 | 16.41972 | 154.4454 | 9.41 |
| C9 | 17.45685 | 160.5407 | 9.20 |
| G9 | 18.56193 | 169.6676 | 9.14 |
| F7 | 17.66779 | 160.8375 | 9.10 |
| D7 | 17.42042 | 156.1915 | 8.97 |
| E4 | 17.39821 | 152.9033 | 8.79 |
| F11 | 18.97592 | 165.9137 | 8.74 |
| D5 | 17.77092 | 153.8732 | 8.66 |
| E3 | 17.88169 | 151.0902 | 8.45 |
| D11 | 17.14419 | 142.5414 | 8.31 |
| D2 | 18.54146 | 153.9568 | 8.30 |
| F8 | 20.23772 | 165.1927 | 8.16 |
| D10 | 18.20894 | 140.9155 | 7.74 |
| F9 | 21.43583 | 165.3577 | 7.71 |
| D9 | 20.99365 | 156.3191 | 7.45 |
| E10 | 21.44788 | 157.24 | 7.33 |
| E8 | 22.89992 | 165.0216 | 7.21 |
| E9 | 23.88629 | 165.4768 | 6.93 |
| H12 | 55.67325 | 49.65198 | 0.89 |
| G12 | 62.11299 | 52.59313 | 0.85 |
| F12 | 66.60268 | 55.74106 | 0.84 |
| E12 | 70.5229 | 54.09381 | 0.77 |

E. coli comprising nucleic acid constructs encoding each of the clones listed in Table 3 as well as GFP S11 (SEQ ID NO: 16) were grown and expression of the possible Split-CFP detector and GFP S11 induced as described above. Split-YFP molecules were detected by detecting yellow and green fluorescence (510/530 nm and 488/510 nm excitation and emission wavelength, respectively) emitted from the bacteria (as described above). As shown in FIGS. 2 and 3, several individual members of the final set of Split-YFP mutants developed using the degenerate library screen described above exhibited distinguishable yellow/green fluorescent properties.

### Split-YFP Optima

The degenerate library screen and the fluorescence assays resulted in the identification of several polypeptides that will complement with GFP S11 (SEQ ID NO: 16) to form a functional Split-YFP molecule. For example, GFP S1-10 with T65L, T203Y, T2055 substitutions (SEQ ID NO: 25) exhibits the greatest yellow to green fluorescence ration for complementation with the SFP tag. GFP 51-10 with T65G, T203Y, T2055 substitutions (SEQ ID NO: 26) exhibits the most spectral exclusion from a SFP formed of GFP 51-10 (SE ID NO: 4) and GFP 5-11 (SEQ ID NO: 16) when complemented with a SFP tag. GFP S1-10 with T203Y and T205A substitutions (SEQ ID NO: 27) exhibits the most fluorescence at the yellow channel (532 nm) when complemented with a SFP tag and excited at 510 nm. An example of the fluorescence of these molecules is shown in FIG. 5.

In view of the many possible embodiments to which the principles of the disclosed invention may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. An isolated polypeptide comprising a Split Fluorescent Protein (SFP) detector comprising an amino acid sequence having at least 95% sequence identity to SEQ ID NO: 23, wherein residues 19 and 21 are E, residue 66 is W, residue 124 is E or V, residue 148 is D, residue 167 is V or I and residue 205 is S, and wherein the SFP detector complements with a SFP tag to form a functional Split-Cyan Fluorescent Protein.

2. The polypeptide of claim 1, comprising an amino acid sequence set forth as SEQ ID NO: 23, SEQ ID NO: 19, SEQ ID NO: 20 or SEQ ID NO: 21.

3. The polypeptide of claim 1 fused to a subcellular localization element.

4. A nucleic acid molecule comprising a nucleotide sequence encoding the polypeptide of claim 1.

5. A host cell comprising the nucleic acid molecule of claim 4.

6. An isolated polypeptide comprising a Split Fluorescent Protein (SFP) detector comprising an amino acid sequence having 95% sequence identity to SEQ ID NO: 31, wherein residue 65 is T, L, G or A, residue 203 is Y, and residue 205 is S, and wherein the SFP detector complements with a SFP tag to form a functional Split-Yellow Fluorescent Protein.

7. The polypeptide of claim 6, comprising an amino acid sequence set forth as SEQ ID NO: 31, SEQ ID NO: 25, SEQ ID NO: 26, SEQ ID NO: 27 or SEQ ID NO: 29.

8. The polypeptide of claim 6 fused to a subcellular localization element.

9. An isolated nucleic acid molecule comprising a nucleotide sequence encoding the polypeptide of claim 6.

10. A host cell comprising the nucleic acid molecule of claim 9.

11. A method of determining a subcellular localization of a protein, comprising:
providing within at least one host cell a first polypeptide comprising a first subcellular localization element and a first Split Fluorescent Protein (SFP) detector comprising a polypeptide comprising an amino acid sequence having at least 95% sequence identity to SEQ ID NO: 23, wherein residues 19 and 21 are E, residue 66 is W, residue 124 is E or V, residue 148 is D, residue 167 is V or I and residue 205 is S and wherein the SFP detector complements with a SFP tag to form a functional Split-Cyan Fluorescent Protein, or an amino acid sequence having 95% sequence identity to SEQ ID NO: 31, wherein residue 65 is T, L, G or A, residue 203 is Y, and residue 205 is S, and wherein the SFP detector complements with a SFP tag to form a functional Split-Yellow Fluorescent Protein, wherein the first subcellular localization element localizes the first polypeptide to a first subcellular compartment;
providing within the host cell a second polypeptide comprising a test protein fused to a SFP tag; and
detecting fluorescence of the first SFP detector complemented with the SFP tag in the host cell, wherein the presence of fluorescence of the first SFP detector complemented with the SFP tag identifies the test protein as localized to the first subcellular compartment, thereby determining a subcellular localization of a protein.

12. The method of claim 11, further comprising:
providing within the host cell a third polypeptide comprising a second subcellular localization element and a second SFP detector, wherein the second subcellular localization element localizes the third polypeptide to a second subcellular compartment, and wherein the second SFP detector can be differentially detected from the first SFP detector when complemented with the SFP tag; and
detecting fluorescence of the second SFP detector complemented with the SFP tag in the host cell, wherein the presence of fluorescence of the second SFP detector complemented with the SFP tag identifies the test protein as localized to the second subcellular compartment.

13. The method of claim 12, wherein the first and third polypeptides comprise any two polypeptides selected from the group consisting of a polypeptide comprising an amino acid sequence having at least 95% sequence identity to SEQ ID NO: 23, wherein residues 19 and 21 are E, residue 66 is W, residue 124 is E or V, residue 148 is D, residue 167 is V or I and residue 205 is S, and wherein the SFP detector complements with a SFP tag to form a functional Split-Cyan Fluorescent Protein, a polypeptide comprising an amino acid sequence having 95% sequence identity to SEQ ID NO: 31, wherein residue 65 is T, L, G or A, residue 203 is Y, and residue 205 is S, and wherein the SFP detector complements with a SFP tag to form a functional Split-Yellow Fluorescent Protein, and a polypeptide comprising a Split-GFP SFP detector.

14. The method of claim 11, wherein detecting SFP fluorescence in the host cell comprises flow cytometry.

15. The method of claim 11, further comprising selecting the host cell that expresses the test protein.

16. The method of claim 11, wherein the test protein is a membrane protein, the SFP tag is fused to the N- or C-terminus of the test protein and the presence of fluorescence of the first SFP detector complemented with the SFP tag in the host cell further identifies the terminus of the test protein fused to the SFP tag as on the same side of the membrane as the first SFP detector.

17. The method of claim 12, wherein the test protein is a membrane protein the SFP tag is fused to the N- or C-terminus of the test protein, the presence of fluorescence of the first SFP detector complemented with the SFP tag in the host cell identifies the terminus of the test protein fused to the SFP tag as on the same side of the membrane as the first SFP detector; and the presence of fluorescence of the second SFP detector complemented with the SFP tag in the host cell identifies the terminus of the test protein fused to the SFP tag as on the same side of the membrane as the second SFP detector.

18. The method of claim 11, wherein providing the first polypeptide or the second polypeptide within the host cell comprises:
expressing the first or second polypeptide within the host cell;
contacting the host cell with the first or second polypeptide; or
a combination thereof.

19. The method of claim 12, wherein providing the first polypeptide, the second polypeptide or the third polypeptide within the host cell comprises:
expressing the first, second or third polypeptide within the host cell;
contacting the host cell with the first, second or third polypeptide; or
a combination thereof.

20. A method for detecting the localization of a test protein to one or more of a plurality of subcellular components in a cell, comprising:
providing within the cell a polypeptide comprising the test protein and a SFP tag;
providing within the cell a plurality of SFP detectors complementary to the SFP tag at least one of which is a polypeptide comprising an amino acid sequence having at least 95% sequence identity to SEQ ID NO: 23, wherein residues 19 and 21 are E, residue 66 is W, residue 124 is E or V, residue 148 is D, residue 167 is V or I and residue 205 is S, and wherein the SFP detector complements with a SFP tag to form a functional Split-Cyan Fluorescent Protein, or an amino acid sequence having 95% sequence identity to SEQ ID NO: 31, wherein residue 65 is T, L, G or A, residue 203 is Y, and residue 205 is S, and wherein the SFP detector complements with a SFP tag to form a functional Split-Yellow Fluorescent Protein, wherein each of the SFP detectors is capable of producing different color fluorescence upon complementation with the SFP tag and each of the SFP detectors is fused to a subcellular localization element that localizes the SFP detector to a different subcellular compartment; and
detecting the various color fluorescence signals in cell, thereby detecting the localization of the test protein to one or more of the subcellular compartments.

21. The method of claim 20, wherein the plurality of SFP detectors comprises:
a polypeptide comprising an amino acid sequence having at least 95% sequence identity to SEQ ID NO: 23, wherein residues 19 and 21 are E, residue 66 is W, residue 124 is E or V, residue 148 is D, residue 167 is V or I and residue 205 is S, and wherein the SFP detector complements with a SFP tag to form a functional Split-Cyan Fluorescent Protein;
a polypeptide comprising an amino acid sequence having 95% sequence identity to SEQ ID NO: 31, wherein residue 65 is T, L, G or A, residue 203 is Y, and residue 205 is S, and wherein the SFP detector complements with a SFP tag to form a functional Split-Yellow Fluorescent Protein;
a Split-GFP SFP detector; or
a combination of two or more thereof.

22. The method of claim 20, wherein detecting SFP fluorescence in the host cell comprises flow cytometry.

23. The method of claim 20, further comprising selecting the host cell that expresses the test protein.

24. The method of claim 20, wherein providing the polypeptide comprising the test protein and the SFP tag or the plurality of SFP detectors within the host cell comprises:
expressing the polypeptide comprising the test protein and the SFP tag or the plurality of SFP detectors within the host cell;
contacting the host cell with the polypeptide comprising test protein and the SFP tag or the plurality of SFP detectors; or
a combination thereof.

25. A method of determining the membrane topology of a membrane protein, comprising:
providing within at least one host cell a first polypeptide comprising a first subcellular localization element and a first Split Fluorescent Protein (SFP) detector comprising a polypeptide comprising an amino acid sequence having at least 95% sequence identity to SEQ ID NO: 23, wherein residues 19 and 21 are E, residue 66 is W, residue 124 is E or V, residue 148 is D, residue 167 is V or I and residue 205 is S, and wherein the SFP detector complements with a SFP tag to form a functional Split-Cyan Fluorescent Protein, or an amino acid sequence having 95% sequence identity to SEQ ID NO: 31, wherein residue 65 is T, L, G or A, residue 203 is Y, and residue 205 is S, and wherein the SFP detector complements with a SFP tag to form a functional Split-Yellow Fluorescent Protein, wherein the first subcellular localization element localizes the first polypeptide to one side of a membrane of the host cell;
providing within the host cell a second polypeptide comprising a test membrane protein, the N- or C-terminus of which is fused to a SFP tag; and
detecting fluorescence of the first SFP detector complemented with the SFP tag in the host cell, wherein the presence of fluorescence of the first SFP detector complemented with the SFP tag in the host cell identifies the membrane orientation of the terminus of test protein fused to the SFP tag as on the same side of the membrane as the first SFP detector, thereby determining the topology of a membrane protein.

26. The method of claim 25, further comprising:
providing within the host cell a third polypeptide comprising a second subcellular localization element and a second Split Fluorescent Protein (SFP) detector, wherein the second subcellular localization element localizes the third polypeptide to the opposite side of membrane of the host cell compared to the first subcellular localization element, and wherein the second SFP detector polypeptide can be differentially detected from the first SFP detector when complemented with the SFP tag; and
detecting fluorescence of the second SFP detector complemented with the SFP tag in the host cell, wherein the presence of fluorescence of the second SFP detector complemented with the SFP tag in the host cell identifies the membrane orientation of the terminus of test protein fused to the SFP tag as on the same side of the membrane as the second SFP detector.

27. The method of claim 26, wherein the first and third polypeptides comprise any two polypeptides selected from the group consisting of a polypeptide comprising an amino acid sequence having at least 95% sequence identity to SEQ ID NO: 23, wherein residues 19 and 21 are E, residue 66 is W, residue 124 is E or V, residue 148 is D, residue 167 is V or I and residue 205 is S, and wherein the SFP detector complements with a SFP tag to form a functional Split-Cyan Fluorescent Protein, a polypeptide comprising an amino acid sequence having 95% sequence identity to SEQ ID NO: 31, wherein residue 65 is T, L, G or A, residue 203 is Y, and residue 205 is S, and wherein the SFP detector complements with a SFP tag to form a functional Split-Yellow Fluorescent Protein, and a polypeptide comprising a Split-GFP SFP detector.

28. The method of claim 25, wherein detecting SFP fluorescence in the host cell comprises flow cytometry.

29. The method of claim 25, further comprising selecting the host cell that expresses the test protein.

30. The method of claim 25, wherein providing the first polypeptide or the second polypeptide within the host cell comprises:
expressing the first or second polypeptide within the host cell;
contacting the host cell with the first or second polypeptide; or
a combination thereof.

31. The method of claim 26, wherein providing the first polypeptide, the second polypeptide or the third polypeptide within the host cell comprises:
expressing the first, second or third polypeptide within the host cell;
contacting the host cell with the first, second or third polypeptide; or
a combination thereof.

32. A kit, comprising:
a nucleic acid construct comprising a nucleic acid molecule encoding a polypeptide comprising an amino acid sequence having at least 95% sequence identity to SEQ ID NO: 23, wherein residues 19 and 21 are E, residue 66 is W, residue 124 is E or V, residue 148 is D, residue 167 is V or I and residue 205 is S, and wherein the SFP detector complements with a SFP tag to form a functional Split-Cyan Fluorescent Protein, or an amino acid sequence having 95% sequence identity to SEQ ID NO: 31, wherein residue 65 is T, L, G or A, residue 203 is Y, and residue 205 is S, and wherein the SFP detector complements with a SFP tag to form a functional Split-Yellow Fluorescent Protein, and a multiple cloning site adjacent thereto, such that an encoding sequence inserted into the multiple cloning site results in a nucleic acid molecule that encodes a protein encoded by the encoding sequence fused with the protein encoded by the nucleic acid molecule; and
instructions for use thereof.

33. A polypeptide comprising a Split Fluorescent Protein (SFP) Detector comprising an amino acid sequence set forth as SEQ ID NO: 22, wherein the SFP detector complements with a SFP tag to form a functional Split-Cyan Fluorescent Protein.

34. A polypeptide comprising a Split Fluorescent Protein (SFP) Detector comprising an amino acid sequence set forth as SEQ ID NO: 30, wherein the SFP detector complements with a SFP tag to form a functional Split-Yellow Fluorescent Protein.


SEQUENCE LISTING (39 sequences)

SEQ ID NO: 1 - DNA, 646 bp, Artificial Sequence
  Variant GFP cDNA
atgagcaaaggagaagaacttttcactggagttgtcccaattcttgttgaattagatggtgatgttaatgggcacaaattttctgtcagaggagagggtgaaggtgatgctacaaacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaaaggtattgattttaaagaagatggaaacattctcggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagctaa

SEQ ID NO: 2 - protein, 214 aa, Artificial Sequence
  Variant GFP protein
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEK

SEQ ID NO: 3 - DNA, 651 bp, Artificial Sequence
  Variant GFP cDNA
atgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcgacacaaactgtcctttcgaaagatcccaacgaaaagggtacctaa

SEQ ID NO: 4 - protein, 216 aa, Artificial Sequence
  Variant GFP protein
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGT

SEQ ID NO: 5 - DNA, 651 bp, Artificial Sequence
  Variant GFP cDNA
atgagcaaaggagaagaacttttcactggagttgtcccaattcttgttgaattagatggagatgttaatgggcacaaattttctgtcagaggagagggtgaaggtgatgctacaaacggaaaactcacccttaaattcatttgcactactggaaaactacctgttccatggccaacgcttgtcactactctgacctatggtgttcaatgcttttcccgttatccggatcacatgaaacagcatgactttttcaagagtgccatgcccgaaggttatgtacaggaacgcactatatatttcaaagatgacgggaactacaagacgcgtgctgtagtcaagtttgaaggtgatacccttgttaatcgtatcgagttaaagggtactgattttaaagaagatggaaacattctcggacacaaactcgagtacaactttaactcacacaatgtatatatcacggcagacaaacaaaagaatggaatcaaagctaacttcacaattcgccacaacgttgtagatggttccgttcaactagcagaccattatcaacaaaatactccaattggcgatggccctgtccttttaccagacaaccattacttgtcgacacaaactgtcctttcgaaagatcccaacgaaaagggtacctaa

SEQ ID NO: 6 - protein, 216 aa, Artificial Sequence
  Variant GFP protein
MSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTIYFKDDGNYKTRAVVKFEGDTLVNRIELKGTDFKEDGNILGHKLEYNFNSHNVYITADKQKNGIKANFTIRHNVVDGSVQLADHYQQNTPIGDGPVLLPDNHYLSTQTVLSKDPNEKGT

SEQ ID NO: 7 - DNA, 84 bp, Artificial Sequence
  Variant GFP cDNA
aagcgtgaccacatggtccttcttgagtttgtaactgctgctgggattacacatggcatggatgagctctacaaaggtacctaa

SEQ ID NO: 8 - protein, 27 aa, Artificial Sequence
  Variant GFP protein
KRDHMVLLEFVTAAGITHGMDELYKGT

SEQ ID NO: 9 - DNA, 60 bp, Artificial Sequence
  Variant GFP cDNA
aagcgtgaccacatggtccttcttgagtttgtaactgctgctgggattacaggtacctaa

SEQ ID NO: 10 - protein, 19 aa, Artificial Sequence
  Variant GFP protein
KRDHMVLLEFVTAAGITGT

SEQ ID NO: 11 - DNA, 60 bp, Artificial Sequence
  Variant GFP cDNA
aagcgtgaccacatggtccttcatgagtttgtaactgctgctgggattacaggtacctaa

SEQ ID NO: 12 - protein, 19 aa, Artificial Sequence
  Variant GFP protein
KRDHMVLHEFVTAAGITGT

SEQ ID NO: 13 - DNA, 54 bp, Artificial Sequence
  Variant GFP cDNA
aagcgtgaccacatggtccttcatgagtctgtaaatgctgctgggggtacctaa

SEQ ID NO: 14 - protein, 17 aa, Artificial Sequence
  Variant GFP protein
KRDHMVLHESVNAAGGT

SEQ ID NO: 15 - DNA, 51 bp, Artificial Sequence
  Variant GFP cDNA
cgtgaccacatggtccttcatgagtctgtaaatgctgctgggattacataa

SEQ ID NO: 16 - protein, 16 aa, Artificial Sequence
  Variant GFP protein
RDHMVLHEYVNAAGIT

SEQ ID NO: 17 - protein, 217 aa, Artificial Sequence
  Variant GFP protein
MSKGEELFTGVVPILVELDGDVNGHKFSVRGEGEGDATIGKLTLKFICTTGKLPVPWPTLVTTLTWGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTSVLSKDPNEKGS

SEQ ID NO: 18 - protein, 216 aa, Artificial Sequence
  Variant GFP protein
MSKGEELFTGVVPILVELDGDVNGHKFSVRGEGEGDATIGKLTLKFICTTGKLPVPWPTLVTTLTWGVQCFSRYPDHMKRHDFFKSAMPEGYVQERTISFKDDGKYKTRAVVKFEGDTLVNRIELKGTDFKEDGNILGHKLEYNFNSDNVYITADKQKNGIKANFTVRHNVEDGSVQLADHYQQNTPIGDGPVLLPDNHYLSTQSVLSKDPNEKGS

SEQ ID NO: 19 - protein, 216 aa, Artificial Sequence
  Variant GFP protein
MSKGEELFTGVVPILVELEGEVNGHKFSVRGEGEGDATIGKLTLKFICTTGKLPVPWPTLVTTLTWGVQCFSRYPDHMKRHDFFKSAMPEGYVQERTISFKDDGKYKTRAVVKFEGDTLVNRIELKGTDFKEDGNILGHKLEYNFNSDNVYITADKQKNGIKANFTVRHNVEDGSVQLADHYQQNTPIGDGPVLLPDNHYLSTQSVLSKDPNEKGS

SEQ ID NO: 20 - protein, 216 aa, Artificial Sequence
  Variant GFP protein
MSKGEELFTGVVPILVELEGEVNGHKFSVRGEGEGDATIGKLTLKFICTTGKLPVPWPTLVTTLTWGVQCFSRYPDHMKRHDFFKSAMPEGYVQERTISFKDDGKYKTRAVVKFEGDTLVNRIVLKGTDFKEDGNILGHKLEYNFNSDNVYITADKQKNGIKANFTVRHNVEDGSVQLADHYQQNTPIGDGPVLLPDNHYLSTQSVLSKDPNEKGS

SEQ ID NO: 21 - protein, 216 aa, Artificial Sequence
  Variant GFP protein
MSKGEELFTGVVPILVELEGEVNGHKFSVRGEGEGDATIGKLTLKFICTTGKLPVPWPTLVTTLTWGVQCFSRYPDHMKRHDFFKSAMPEGYVQERTISFKDDGKYKTRAVVKFEGDTLVNRIELKGTDFKEDGNILGHKLEYNFNSDNVYITADKQKNGIKANFTIRHNVEDGSVQLADHYQQNTPIGDGPVLLPDNHYLSTQSVLSKDPNEKGS

SEQ ID NO: 22 - protein, 216 aa, Artificial Sequence
  Variant GFP protein
MSKGEELFTGVVPILXELXGXVNGHKFSVRGEGEGDATIGKLTLKFICTTGKLPVPWPTLVTTLTWGVQCFSRYPDHMKRHDFFKSAMPEGYVQERTIXFKDDGKYKTRAVVKFEGDTLVNRIXLKGTDFKEDGNILGHKLEYNFNSDNVYITADKQKNGIKANFTXRHNVEDGSVQLADHYQQNTPIGDGPVLLPDNHYLSTQSVLSKDPNEKGS

SEQ ID NO: 23 - protein, 216 aa, Artificial Sequence
  Variant GFP protein
MSKGEELFTGVVPILVELEGEVNGHKFSVRGEGEGDATIGKLTLKFICTTGKLPVPWPTLVTTLTWGVQCFSRYPDHMKRHDFFKSAMPEGYVQERTISFKDDGKYKTRAVVKFEGDTLVNRIXLKGTDFKEDGNILGHKLEYNFNSDNVYITADKQKNGIKANFTXRHNVEDGSVQLADHYQQNTPIGDGPVLLPDNHYLSTQSVLSKDPNEKGS

SEQ ID NO: 24 - protein, 216 aa, Artificial Sequence
  Variant GFP protein
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSYQTVLSKDPNEKGS

SEQ ID NO: 25 - protein, 216 aa, Artificial Sequence
  Variant GFP protein
MSKGEELFTGVVPILVELDGDVNGHKFSVRGEGEGDATIGKLTLKFICTTGKLPVPWPTLVTTLLYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSYQSVLSKDPNEKGS

SEQ ID NO: 26 - protein, 216 aa, Artificial Sequence
  Variant GFP protein
MSKGEELFTGVVPILVELDGDVNGHKFSVRGEGEGDATIGKLTLKFICTTGKLPVPWPTLVTTLGYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSYQSVLSKDPNEKGS

SEQ ID NO: 27 - protein, 216 aa, Artificial Sequence
  Variant GFP protein
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSYQSVLSKDPNEKGS

SEQ ID NO: 28 - protein, 216 aa, Artificial Sequence
  Variant GFP protein
MSKGEELFTGVVPILVELDGDVNGHKFSVRGEGEGDATIGKLTLKFICTTGKLPVPWPTLVTTLAYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSYQSVLSKDPNEKGS

SEQ ID NO: 29 - protein, 216 aa, Artificial Sequence
  Variant GFP protein
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSYQAVLSKDPNEKGS

SEQ ID NO: 30 - protein, 216 aa, Artificial Sequence
  Variant GFP protein
MSKGEELFXGVVPILVELDGDVNGHKFSVRGEGEGDATIGKLTLKFICTTGKLPVPWPTLVTTLXYGVQXFSRYPDHMKXHDFFKSAMPEGYVQERTIXFKDDGKYKTRAVVKFEGDTLVNRIELKGTDFKEDGNILGHKLEYNFNSHNVYITADKQKNGIKANFTVRHNVEDGSXQLADHYQQNTPIGDGXVLLPDNHXLSYXXVLSKXPNEKGS

SEQ ID NO: 31 - protein, 216 aa, Artificial Sequence
  Variant GFP protein
MSKGEELFTGVVPILVELDGDVNGHKFSVRGEGEGDATIGKLTLKFICTTGKLPVPWPTLVTTLXYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSYQXVLSKDPNEKGS

SEQ ID NO: 32 - protein, 28 aa, Artificial Sequence
  Nuclear localization signal protein
SKKEEKGRSKKEEKGRSKKEEKGRIHRI

SEQ ID NO: 33 - DNA, 87 bp, Artificial Sequence
  Nuclear localization signal cDNA
tccaaaaaagaagagaaaggtagatccaaaaaagaagagaaaggtagatccaaaaaagaagagaaaggtaggatccaccggatctag

SEQ ID NO: 34 - protein, 89 aa, Artificial Sequence
  human beta 1,4-galactosyltransferase N-terminus protein
MRLREPLLSGSAAMPGASLQRACRLLVAVCALHLGVTLVYYLAGRDLSRLPQLVGVSTPLQGGSNSAAAIGQSSGELRTGGAKDPPVAT

SEQ ID NO: 35 - DNA, 267 bp, Artificial Sequence
  human beta 1,4-galactosyltransferase N-terminus cDNA
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggccaaggatccaccggtcgccacc

SEQ ID NO: 36 - protein, 36 aa, Artificial Sequence
  cytochrome C oxidase fragment protein
MSVLTPLLLRGLTGSARRLPVPRAKIHSLGDPPVAT

SEQ ID NO: 37 - DNA, 108 bp, Artificial Sequence
  cytochrome C oxidase cDNA fragment
atgtccgtcctgacgccgctgctgctgcggggcttgacaggctcggcccggcggctcccagtgccgcgcgccaagatccattcgttgggggatccaccggtcgccacc

SEQ ID NO: 38 - protein, 18 aa, Artificial Sequence
  ER targeting sequence of calreticulin protein
MLLSVPLLLGLLGLAVAV

SEQ ID NO: 39 - DNA, 54 bp, Artificial Sequence
  ER targeting sequence of calreticulin cDNA
atgctgctatccgtgccgttgctgctcggcctcctcggcctggccgtcgccgtg